(19) United States
(12) Patent Application Publication
(10) Pub. No.: US 2011/0160550 A1
(43) Pub. Date: Jun. 30, 2011

(54) METHOD FOR TAGGING CONDITION INFORMATION AND MULTIMEDIA APPARATUS USING THE SAME

(75) Inventors: Ho-ik HWANG, Hwaseong-si (KR); Soo-hyun LEE, Seoul (KR); Se-jin KWAK, Seoul (KR); Byeong-cheol HWANG, Seoul (KR); Joon-ho WON, Suwon-si (KR)

(73) Assignee: SAMSUNG ELECTRONICS CO. LTD., Suwon-si (KR)

(21) Appl. No.: 12/974,448

(22) Filed: Dec. 21, 2010

(30) Foreign Application Priority Data
Dec. 24, 2009 (KR) 10-2009-0130487

(51) Int. Cl. A61B 6/00; G06F 17/30

(52) U.S. Cl. 600/301; 707/741; 707/E17.002

(57) ABSTRACT

A method for tagging condition information and a multimedia apparatus using the same are provided. The method includes measuring condition information of a user who is enjoying content, and tagging the content with the measured condition information. Accordingly, the user's condition information for each content is checked and the user's condition information is adjusted using the condition information tagged onto the contents.

### PRIORITY

This application claims the benefit under 35 U.S.C. §119(a) of a Korean patent application filed on Dec. 24, 2009 in the Korean Intellectual Property Office and assigned Serial No. 10-2009-0130487, the entire disclosure of which is hereby incorporated by reference.

### BACKGROUND OF THE INVENTION

1. Field of the Invention

The present invention relates to a method for tagging condition information and a multimedia apparatus using the same. More particularly, the present invention relates to a method for tagging condition information, which measures user's condition information and tags content with the measured condition information, and a multimedia apparatus using the same.

2. Description of the Related Art

In recent years, with the proliferation of multimedia apparatuses, the amount of content used in a multimedia apparatus has increased substantially. Based on the increased amount of content, users are able to select and use more diverse content.

Typically, content is tagged with information. For example, a music file may be tagged with information regarding a composer, an album title, a genre, and the like. Accordingly, the user can be provided with a list having content sorted according to the tagged information or can select content according to the tagged information.

However, even if such tagged information is used, a user must input a sorting or selecting command that is an inconvenience for the user. Also, the multimedia apparatus is unable to provide a list of content using the tagged information.

Therefore, there is a need for a multimedia apparatus to provide a list of content to a user using tagged information.

### SUMMARY OF THE INVENTION

An aspect of the present invention is to address at least the above-mentioned problems and/or disadvantages and to provide at least the advantages described below. Accordingly, an aspect of the present invention is to provide a method for tagging content with condition information in order for a user to use the condition information for the content, and a multimedia apparatus using the same.

According to an aspect of the present invention, a method for tagging condition information is provided. The method includes, if content selected from a plurality of contents is being played, determining condition information of a user, and tagging the content with the determined condition information.

According to another aspect of the present invention, a multimedia apparatus is provided. The apparatus includes, a sensor module for, if content selected from a plurality of contents is being played, measuring condition information of a user, and a controller for tagging the content with the measured condition information.

Other aspects, advantages, and salient features of the invention will become apparent to those skilled in the art from the following detailed description, which, taken in conjunction with the annexed drawings, discloses exemplary embodiments of the invention.

### BRIEF DESCRIPTION OF THE DRAWING FIGURES

The above and other aspects, features, and advantages of certain exemplary embodiments of the present invention will be more apparent from the following description taken in conjunction with the accompanying drawings, in which:

FIG. 1 is a view illustrating an MPEG audio layer-3 (MP3) player according to an exemplary embodiment of the present invention;

FIG. 2 is a view illustrating an MP3 player according to an exemplary embodiment of the present invention;

FIG. 3 is a view illustrating a menu structure of an MP3 player according to an exemplary embodiment of the present invention;

FIGS. 4A to 4E are views illustrating screens for selecting a main menu according to an exemplary embodiment of the present invention;

FIGS. 5A and 5B are views illustrating screen configurations according to gender according to an exemplary embodiment of the present invention;

FIG. 6 is a view illustrating a menu screen when a menu of ‘Media’ is selected according to an exemplary embodiment of the present invention;

FIGS. 7A to 7C are views illustrating screens when a menu of ‘Music’ is selected according to an exemplary embodiment of the present invention;

FIGS. 8A and 8B are views illustrating screens when a menu of ‘Videos’ is selected according to an exemplary embodiment of the present invention;

FIG. 9 is a view illustrating a screen when a menu of ‘FM Radio’ is selected according to an exemplary embodiment of the present invention;

FIG. 10 is a view illustrating a screen when a menu of ‘Pictures’ is selected according to an exemplary embodiment of the present invention;

FIGS. 11A to 11C are views illustrating a screen when a menu of ‘Settings’ is selected according to an exemplary embodiment of the present invention;

FIG. 12 is a view illustrating a menu screen when a menu of ‘Biocheck’ is selected according to an exemplary embodiment of the present invention;

FIGS. 13A to 13E are views illustrating screens when a menu of ‘Fat’ is selected according to an exemplary embodiment of the present invention;

FIGS. 14A to 14E are views illustrating screens when a menu of ‘Stress’ is selected according to an exemplary embodiment of the present invention;

FIGS. 15A to 15C are views illustrating screens when a menu of ‘Skin Color’ is selected according to an exemplary embodiment of the present invention;

FIG. 16 is a view illustrating a menu screen when a menu of ‘Fitness’ is selected according to an exemplary embodiment of the present invention;

FIGS. 17A to 17C are views illustrating screens when a menu of ‘Jogging’ is selected according to an exemplary embodiment of the present invention;

FIGS. 18A to 18N are views illustrating screens when a menu of ‘My Coach’ is selected according to an exemplary embodiment of the present invention;

FIG. 19 is a view illustrating a menu screen when a menu of ‘Pedometer’ is selected according to an exemplary embodiment of the present invention;

FIG. 20 is a view illustrating a menu screen when a menu of ‘My Log’ is selected according to an exemplary embodiment of the present invention;

FIGS. 21A and 21B are views illustrating screens when a menu of ‘Today's Summary’ is selected according to an exemplary embodiment of the present invention;

FIGS. 22A to 22C are views illustrating screens when a menu of ‘Exercise Diary’ is selected according to an exemplary embodiment of the present invention;

FIGS. 23A to 23D are views illustrating screens when a menu of ‘Food Diary’ is selected according to an exemplary embodiment of the present invention;

FIGS. 24A to 24C are views illustrating screens when a menu of ‘Calorie Manager’ is selected according to an exemplary embodiment of the present invention;

FIG. 25 is a view illustrating an example of a music file that is tagged with information regarding a health condition or a fitness condition of a user according to an exemplary embodiment of the present invention;

FIGS. 26A and 26B are views illustrating a method of using tagged information according to an exemplary embodiment of the present invention;

FIGS. 27A to 27E are views illustrating a method of using tagged information according to an exemplary embodiment of the present invention;

FIG. 28 is a flowchart illustrating a method for providing a play list according to an exemplary embodiment of the present invention;

FIG. 29 is a view illustrating an MP3 player according to an exemplary embodiment of the present invention; and

FIG. 30 is a view illustrating a sensor module according to an exemplary embodiment of the present invention.

Throughout the drawings, it should be noted that like reference numbers are used to depict the same or similar elements, features, and structures.

### DETAILED DESCRIPTION OF EXEMPLARY EMBODIMENTS

The following description with reference to the accompanying drawings is provided to assist in a comprehensive understanding of the exemplary embodiments of the invention as defined by the claims and their equivalents. It includes various specific details to assist in that understanding but these are to be regarded as merely exemplary. Accordingly, those of ordinary skill in the art will recognize that various changes and modifications of the embodiments described herein can be made without departing from the scope and spirit of the invention. In addition, descriptions of well-known functions and constructions may be omitted for clarity and conciseness.

The terms and words used in the following description and claims are not limited to the bibliographical meanings, but, are merely used by the inventor to enable a clear and consistent understanding of the invention. Accordingly, it should be apparent to those skilled in the art that the following description of exemplary embodiments of the present invention is provided for illustration purpose only and not for the purpose of limiting the invention as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

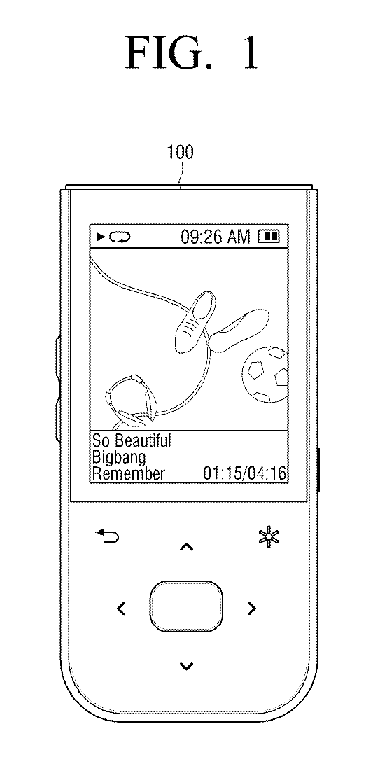

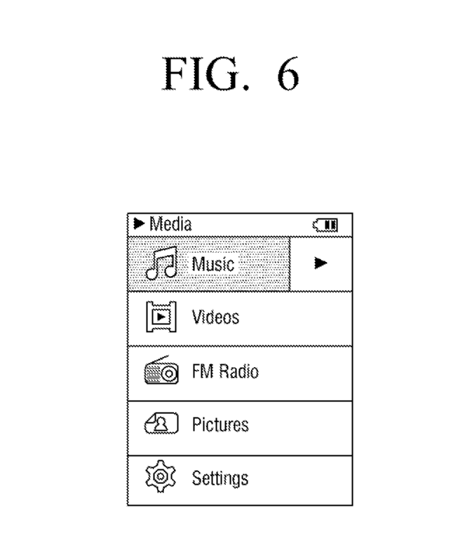

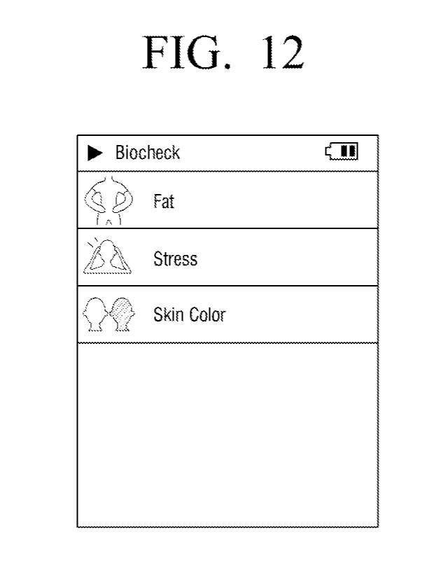

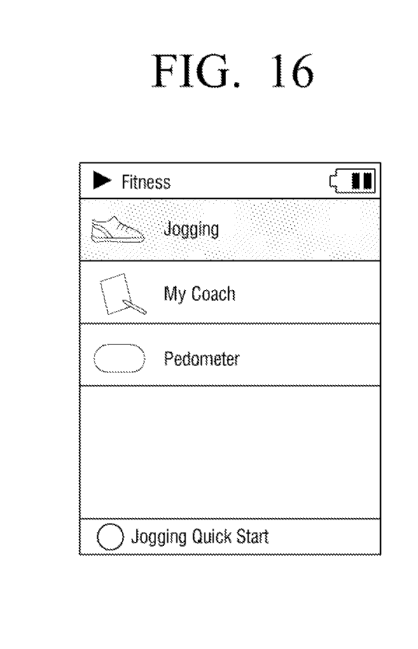

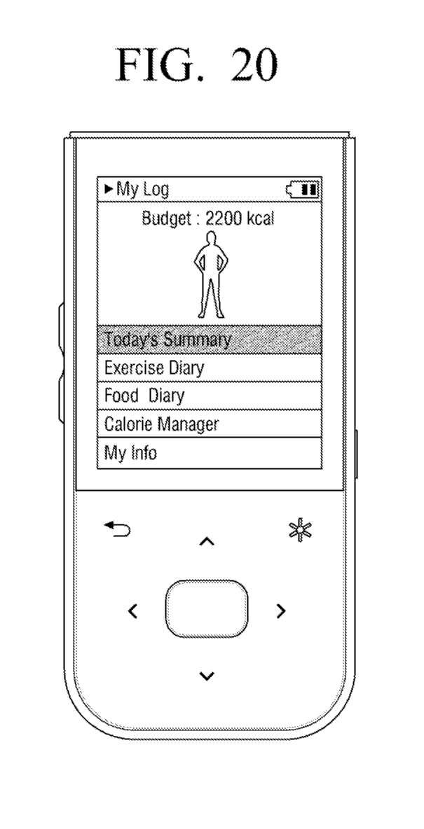

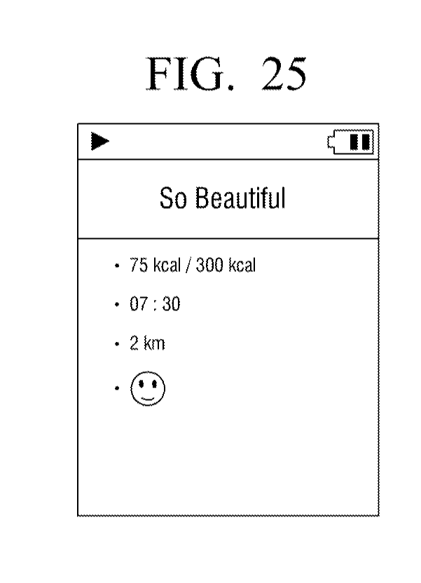

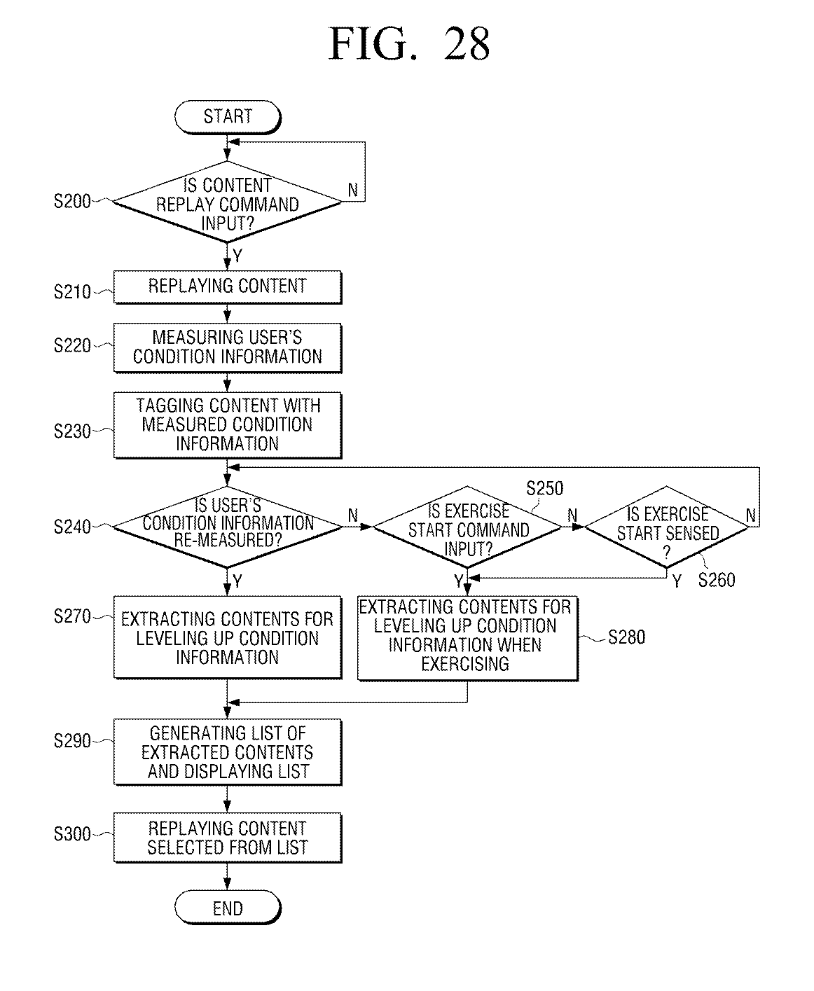

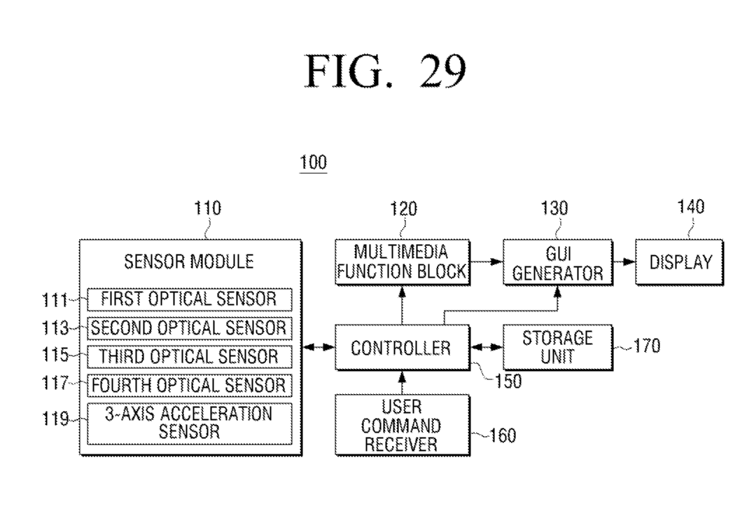

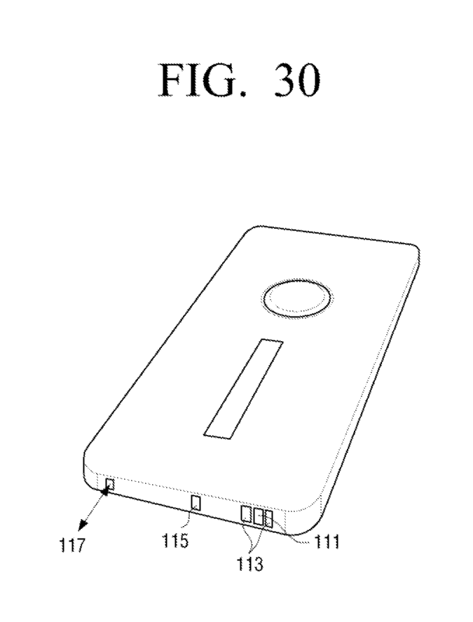

The following will be explained:
1. Overall operation of an MPEG audio layer-3 (MP3) player (FIGS. 1 to 5B)
2. Functions relating to a ‘Media’ menu (FIGS. 6 to 11C)
3. Functions relating to a ‘Biocheck’ menu (FIGS. 12 to 15C)
4. Functions relating to a ‘Fitness’ menu (FIGS. 16 to 19)
5. Functions relating to a ‘My Log’ menu (FIGS. 20 to 24C)
6. Method of generating and using tagged information (FIGS. 25 to 27E)
7. Flowchart for providing a play list (FIG. 28)
8. Configuration of an MP3 player (FIGS. 29 and 30)

### 1. Overall Operation of an MP3 Player

Hereinafter, an overall operation of an MP3 player will be explained with reference to FIGS. 1 to 5B.

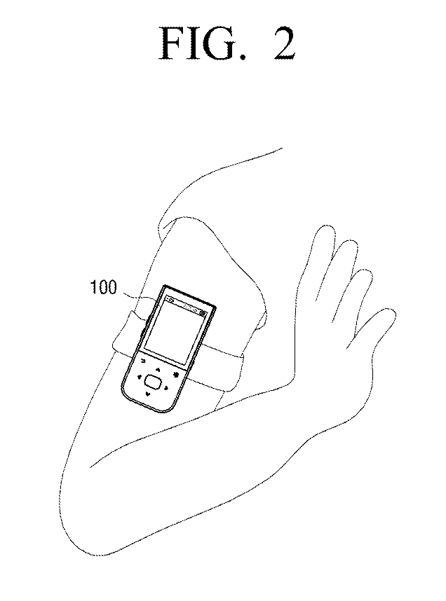

FIGS. 1 and 2 are views illustrating an MP3 player according to an exemplary embodiment of the present invention. More particular, FIG. 1 illustrates only an MP3 player and FIG. 2 illustrates an MP3 player worn on a user's arm using an armband.

Referring to FIG. 1, the MP3 player 100 is a multimedia apparatus for replaying a music file or a video file and providing it to the user. Furthermore, the MP3 player 100 not only replays multimedia files but also determines and manages a user's condition and determines/manages a schedule relating to the user's condition.

The user's condition refers to any condition regarding a user's such as the user's health condition or fitness condition.

The health condition is a concept including any item relating to the user's health, such as amounts of subcutaneous fat or body fat, which are criteria for measuring obesity, stress index, skin color, heart rate, blood pressure, blood sugar, weight, and the like. The fitness condition is a concept including any item relating to a user's exercise such as the number of steps taken during exercise, movement speed, the number of exercises, the calories burned during exercise, and the like.

The MP3 player 100 displays the replayed multimedia content and the user's health condition, fitness condition, and schedule.

The MP3 player 100 includes various keys formed on a front portion and a side portion, for receiving a user's manipulation. Any of the keys may be used as a hot key for changing a mode of the MP3 player 100 to a mode for checking and managing the user's health condition, and any other key may be used as a hot key for changing a mode of the MP3 player 100 to a mode for checking and managing the user's fitness condition.

The MP3 player 100 includes an optical sensor, formed on an upper end portion thereof, for collecting condition information relating to the user's health condition and the user's fitness condition. The MP3 player 100 also includes a 3-axis acceleration sensor for collecting condition information relating to the user's fitness condition.

The optical sensor and the 3-axis acceleration sensor will be explained later in more detail.

The MP3 player may be used independently for replaying a multimedia file, checking and managing the user's health condition, and checking and managing the user's schedule, as shown in FIG. 1, or may be used along with the armband for checking and managing the user's fitness condition as shown in FIG. 2.

In the case that the MP3 player 100 is used independently as shown in FIG. 1, it is possible to manipulate the MP3 player 100 using the above-described keys, and, in the case that the MP3 player is used along with the armband as shown in FIG. 2, it is possible to manipulate the MP3 player 100 by tapping the MP3 player 100 worn on the user's arm using the armband.

For example, tapping the MP3 player 100 one time may cause the MP3 player to play or pause a multimedia file, tapping the MP3 player 100 two times at a time may cause the MP3 player to play a music file of a next track, and tapping the MP3 player three times at a time may cause the MP3 player to add a current music file to a play list.

Of course, the above tapping manipulations are merely an example for convenience of explanation and other functions may be set to correlate with the tapping manipulations.

Also, the tapping manipulation is not limited to the case that the MP3 player 100 is used along with the armband. The tapping manipulation is possible when only the MP3 player 100 is used. Furthermore, in the case that the MP3 player 100 is used along with the armband, the manipulation using the keys is possible.

Also, the tapping manipulation of the MP3 player 100 may be set to be recognized when vibration on the MP3 player 100 is detected by the 3-axis sensor or when a touch on a screen of the MP3 player 100 is detected by a touch detection module.

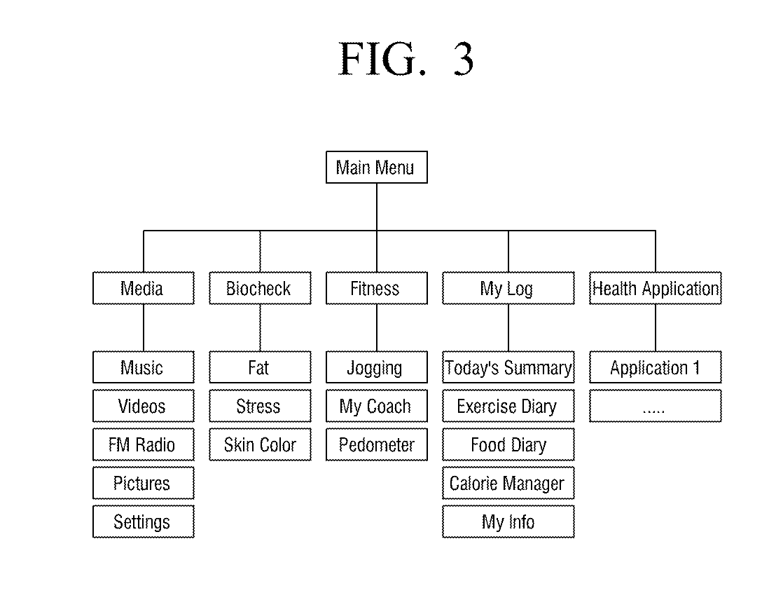

FIG. 3 is a view illustrating a menu structure of an MP3 player according to an exemplary embodiment of the present invention.

Referring to FIG. 3, the MP3 player includes five main menus such as ‘Media’, ‘Biocheck’, ‘Fitness’, My Log', and ‘Health Application’, and each main menu includes sub-menus.

The menu of ‘Media’ relates to the replaying of multimedia files as described above. The user may replay a music file or a video file or manage a list of files to be replayed by manipulating the sub-menus under the menu of ‘Media’.

The menu of ‘Biocheck’ relates to checking and managing the user's health condition as described above. The user may measure his/her own obesity for each body part, a degree of stress, and skin color by manipulating the sub-menus under the menu of ‘Biocheck’.

The menu of ‘Fitness’ relates to checking and managing the user's fitness condition as described above. The user may check information of calories burned during exercise or receive a prescription for the exercise to burn calories, by manipulating the sub-menus under the menu of ‘Fitness’.

The menu of ‘My Log’ relates to managing the user's schedule as described above. The schedule recited herein refers to not only a user's work schedule but also a schedule for achieving a user's target health condition or user's target fitness condition. The user may control the number of exercises or an amount of food by manipulating the sub-menus under the menu of ‘My Diary’.

The menu of ‘Health Application’ relates to executing applications and programs stored in an internal repository of the MP3 player 100 or applications and programs received from an external source and stored in the internal repository. More particularly, the menu of ‘Health Application’ relates to content regarding the user's health or exercise among the applications and the programs stored in the internal repository. The menu of ‘Health Application’ has the application and the program as its sub-menus.

More specifically, the menu of ‘Media’ includes five sub-menus, ‘Music’, ‘Videos’, ‘FM Radio’, ‘Picture’, and ‘Settings’, and the menu of ‘Biocheck’ includes three sub-menus, ‘Fat’, ‘Stress’, and ‘Skin Color’. The menu of ‘Fitness’ includes three sub-menus, ‘Jogging’, ‘My Coach’, and ‘Pedometer’, and the menu of ‘My Log’ includes five sub-menus, ‘Today's Summary’, ‘Exercise Diary’, ‘Food Diary’, ‘Calorie Manager’, and ‘My Info’. The menu of ‘Health Application’ includes one or more sub-menus according to the user's setting as described above.

A more detailed operation of the MP3 player according to manipulation of each sub-menu will be explained later.

The above-described menu structure is merely an example for convenience of explanation and the details of the main menus and the sub-menus are not limited to the above-described details. Also, relationships between the main menus and the sub-menus are not limited the above-described relationships.

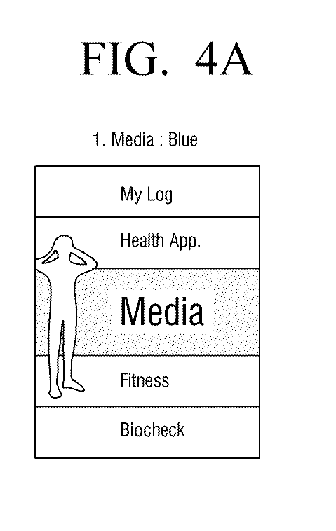

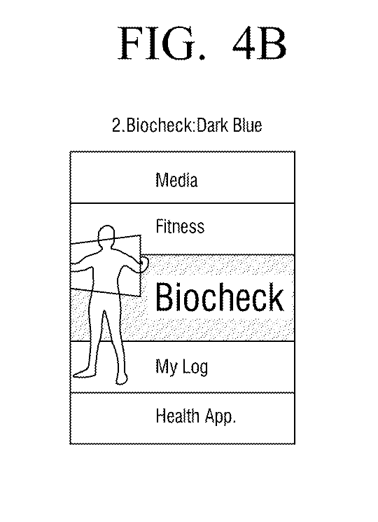

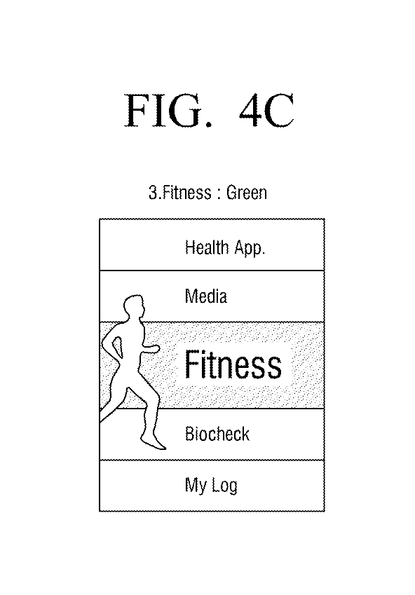

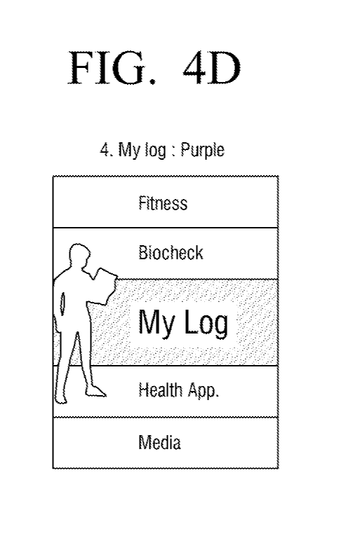

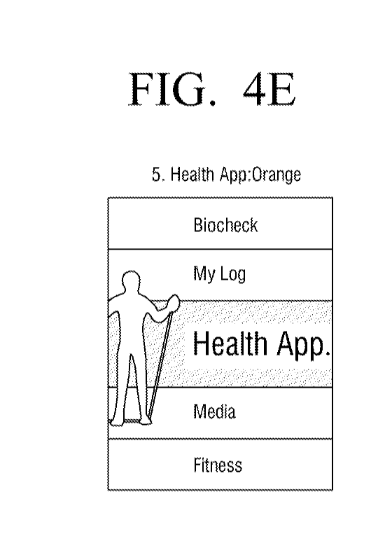

FIGS. 4A to 4E are views illustrating screens for selecting one of the above-described main menus according to exemplary embodiments of the present invention.

FIG. 4A illustrates a screen for selecting the menu of ‘Media’ according to an exemplary embodiment of the present invention. The menu of ‘Media’ is selected by selecting an item of ‘Media’ from among a plurality of items displayed on the screen.

Referring to FIG. 4A, if a condition for selection of the item of ‘Media’ is prepared by a user's manipulation, all items including the item of ‘Media are changed to blue color and the item of ‘Media’ is highlighted. Of course, the blue color is merely an example and any other color may be selected or applied.

The condition for the selection of the item of ‘Media’ refers to a pre-step before the item of ‘Media’ is selected and refers to a condition under which a cursor, a highlight, or an indicator is placed on the item of ‘Media’. In other words, if an OK button on the MP3 player 100 is selected under the condition for the selection of the item of ‘Media’, the item of ‘Media’ is selected.

As described above, when the condition for the selection of the item of ‘Media’ is prepared, the MP3 player 100 changes all items including the item of ‘Media’ to blue color, so that the user can know intuitively which menu he or she can select.

Also, a character item relating to the menu prepared for the selection is displayed on the left portion of the menu screen. The character items are displayed as graphics relating to the menu prepared for the selection, so that the user can know more intuitively which menu he or she can select.

The above operation may also be applied to a case where other menus are selected.

FIG. 4B illustrates a screen for selecting the menu of ‘Biocheck’ according to an exemplary embodiment of the present invention.

Referring to FIG. 4B, if a condition for selection of the item of ‘Biocheck’ is prepared by a user's manipulation, all items including the item of ‘Biocheck are changed to dark blue color and the item of ‘Biocheck’ is highlighted. Also, a character item relating to the menu of ‘Biocheck’ is displayed on the left portion of the menu screen.

FIG. 4C illustrates a screen for selecting the menu of ‘Fitness’ according to an exemplary embodiment of the present invention.

Referring to FIG. 4C, if a condition for selection of the item of ‘Fitness’ is prepared by a user's manipulation, all items including the item of ‘Fitness’ are changed to green color and the item of ‘Fitness’ is highlighted. Also, a character item relating to the menu of ‘Fitness’ is displayed on the left portion of the menu screen.

FIG. 4D illustrates a screen for selecting the menu of ‘My Log’ according to an exemplary embodiment of the present invention.

Referring to FIG. 4D, if a condition for selection of the item of ‘My Log’ is prepared by a user's manipulation, all items including the item of ‘May Log’ are changed to purple color and the item of ‘My Log’ is highlighted. Also, a character item relating to the menu of ‘My Log’ is displayed on the left portion of the menu screen.

FIG. 4E illustrates a screen for selecting the menu of ‘Health Application’ according to an exemplary embodiment of the present invention.

Referring to FIG. 4E, if a condition for selection of the item of ‘Health Application’ is prepared by a user's manipulation, all items including the item of ‘Health Application’ are changed to orange color and the item of ‘Health Application’ is highlighted. Also, a character item relating to the menu of ‘Health Application’ is displayed on the left portion of the menu screen.

As described above, when the condition for selection of the menu is prepared, the MP3 player changes the color of the entire screen according to the menu prepared for the selection and also displays the character item, so that the user can know intuitively which menu he or she can select. Of course, the colors described as associated with each menu are merely for example and not to be construed as limiting. That is, any color may be used for any of the menus.

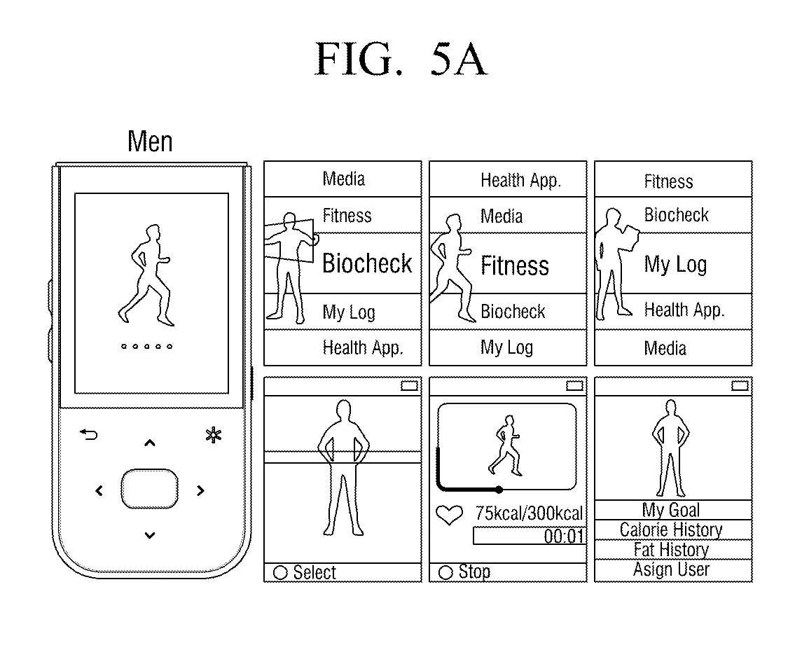

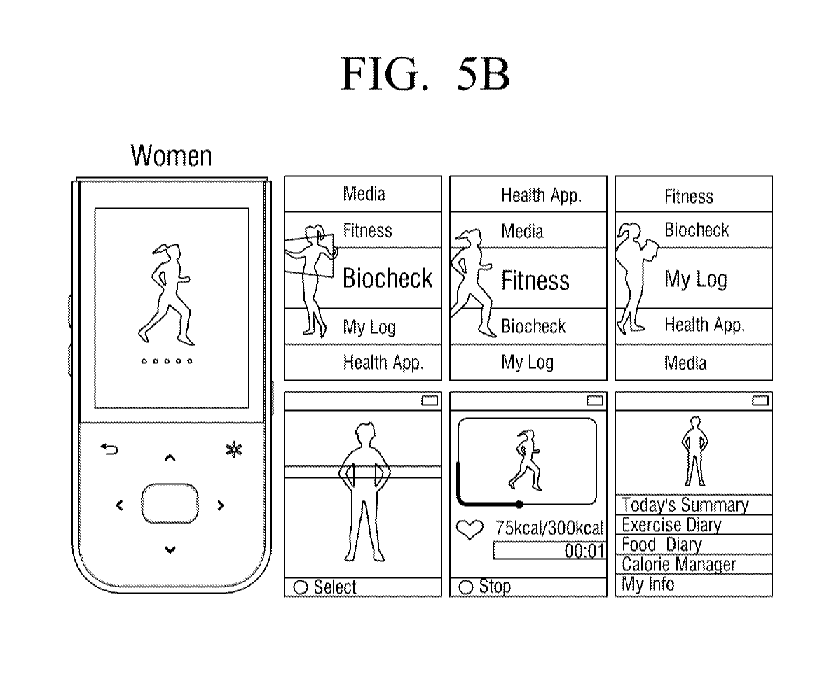

FIGS. 5A and 5B are views illustrating screen configuration according to gender according to an exemplary embodiment of the present invention.

As described above, an MP3 player is able to perform checking and managing the user's health condition and checking and managing the user's fitness condition. The health condition and the fitness condition may be measured or managed in different ways according to user's gender.

Accordingly, the MP3 player checks and manages the health condition and checks and manages the fitness condition according to the user's gender. For example, if the user is a man, the MP3 player is able to prescribe exercise suitable for the man and calculate reference calories suitable for the man.

Referring to FIGS. 5A and 5B, the MP3 player may display different menu screens according to the user's gender. In other words, if the user of the MP3 player 100 is a man, a male character item is displayed as shown in FIG. 5A, and, if the user of the MP3 player 100 is a woman, a female character item is displayed as shown in FIG. 5B.

As described above, the health condition and the fitness condition are checked and managed in different ways according to the user's gender so that more accurate information about the health condition and the fitness condition can be provided to the user. Also, different screens are provided according to the user's gender so that the user's interest on the health and exercise can be increased.

### 2. Functions Relating to Menu of ‘Media’

Hereinafter, functions of an MP3 player relating to the menu of ‘Media’ will be described with reference to FIGS. 6 to 11C.

FIG. 6 is a view illustrating a menu screen when a menu of ‘Media’ is selected according to an exemplary embodiment of the present invention. As shown in FIG. 6, the menu of ‘Media’ includes the five sub-menus, ‘Music’, ‘Videos’, ‘FM Radio’, ‘Pictures’ and ‘Settings’.

Referring to FIG. 6, each sub-menu includes a graphic item and a text item for indicating a type of each sub-menu. For example, the sub-menu of ‘Music’ includes a graphic item ( [symbol] ) and a text item ('Music') for indicating the menu of ‘Music’.

As described above, the graphic item and the text item are displayed so that the user can more intuitively recognize the type of a corresponding menu.

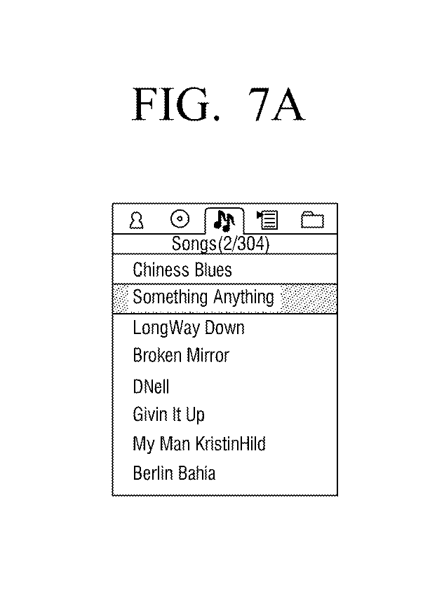

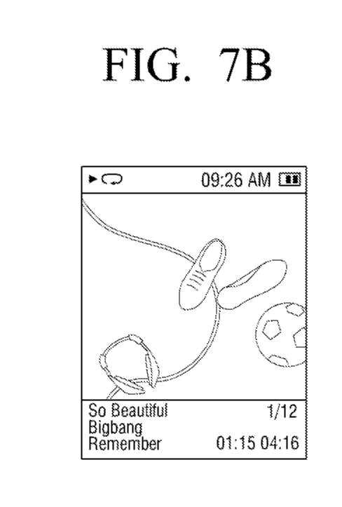

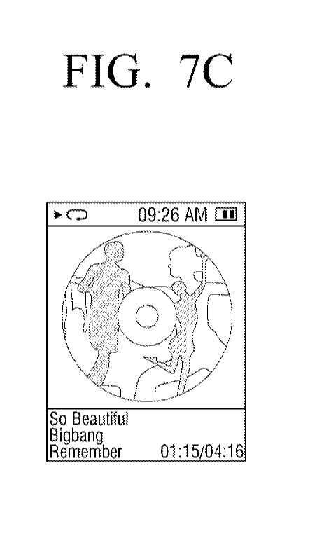

FIGS. 7A to 7C are views illustrating screens when a menu of ‘Music’ is selected according to an exemplary embodiment of the present invention.

Referring to FIG. 7A, if the user selects the menu of ‘Music’, a play list regarding a music file is displayed.

The play list may have music classified by artiest, album, title, genre, and composer, and may be displayed according to the categories when a tab that is displayed on the upper portion of the screen is selected. In other words, the play list may be a list of music files that are included in the menu of ‘Music’ selected by the user and classified by the MP3 player.

Also, the play list may be a list of music files that are stored in the MP3 player and classified according to the user's preference.

If a condition for selection of music from the displayed play list is prepared, an item corresponding to the music prepared for the selection is highlighted. In FIG. 7A, an item corresponding to music titled ‘Something Anything’ is highlighted.

If music to be replayed is selected by a manipulation for the selection of a corresponding music file, the selected music is replayed and an album art corresponding to the replayed music is displayed. In FIG. 7B, the music titled ‘So Beautiful’ is selected and an album art corresponding to the music ‘So Beautiful’ is displayed.

Such an album art is matched with each music file and stored in the internal repository of the MP3 player or is tagged onto a music file and stored along with the music file. Therefore, if a replay command regarding corresponding music is input, the music is replayed and simultaneously an album art matched with or tagged onto the music file is displayed on the screen.

All music is not matched with or tagged with corresponding album art. In some situations, the album art may be neither matched with nor tagged onto the music. Therefore, if a replay command regarding music which is neither matched nor tagged with an album art is input, an album art stored as a default may be displayed when the corresponding music is replayed, as shown in FIG. 7C.

One default album art may be stored in the internal repository of the MP3 player or a plurality of default album arts may be stored in the internal repository of the MP3 player. If the plurality of default album arts are provided, the MP3 player controls the default album art to be displayed on the screen according to a specific regulation or randomly.

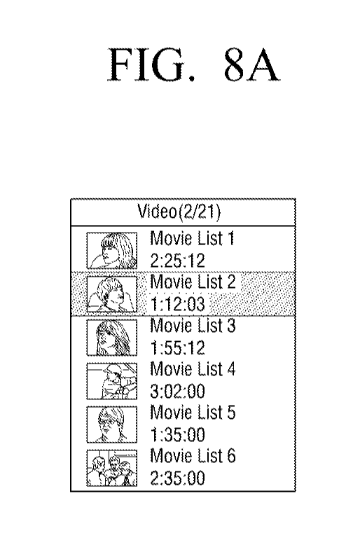

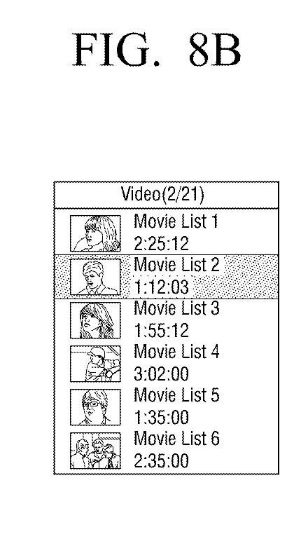

FIGS. 8A and 8B are views illustrating screens when a menu of ‘Videos’ is selected according to an exemplary embodiment of the present invention.

Referring to FIG. 8A, if the user selects the menu of ‘Videos’, a play list regarding moving picture files is displayed.

The play list has moving picture files listed in the order of creation time, replay duration, recently replayed files, and the like.

The play list may be a list of items corresponding to the moving picture files, and, as shown in FIG. 8A, each item may include a thumbnail image, a title, and the replay duration of each moving picture file.

The thumbnail image may be an image corresponding to an initial frame of the moving picture file or an image designated by the user.

Also, if a condition for selection of one of the images included in the displayed play list is prepared, an item corresponding to an image prepared for the selection is highlighted. In FIG. 8A, an item corresponding to an image of ‘Movie List 2’ is highlighted. If the image to be replayed is selected by a manipulation for the selection of the image, the selected image is replayed on the screen.

As noted above, the thumbnail image of FIG. 8A may be an image corresponding to the initial frame or an image designated by the user. However, the thumbnail image may also be another image.

Referring to FIG. 8B, there is illustrated an image corresponding to a pausing time at which the user stops viewing a moving picture, as a thumbnail image. Comparing the screen of FIG. 8B and the screen of FIG. 8A, the thumbnail image corresponding to the moving picture of ‘Movie List 2’ is changed.

When the user stops viewing a moving picture halfway, the user may wish to review the moving picture again at a later time. The MP3 player displays an image corresponding to the time at which the user stops viewing the moving picture as a thumbnail image, so that the thumbnail image serves as a kind of bookmark.

Accordingly, the user may be reminded of a previous image or part that he or she viewed before a replay command for a moving picture is input.

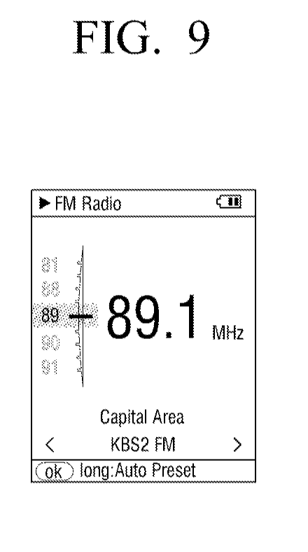

FIG. 9 is a view illustrating a screen when a menu of ‘FM Radio’ is selected according to an exemplary embodiment of the present invention.

Referring to FIG. 9 if the user selects the menu of ‘FM Radio’, frequency information is displayed.

The frequency information is a concept including information regarding entire frequency bands as shown in the left portion of FIG. 9, information regarding a currently tuned frequency as shown in the middle portion of FIG. 9, and information regarding an area set for tuning and a broadcasting station using the tuned frequency as shown in the lower portion of FIG. 9.

Also, the MP3 player outputs a radio broadcast received at the currently tuned frequency at the same time of displaying the frequency information on the screen.

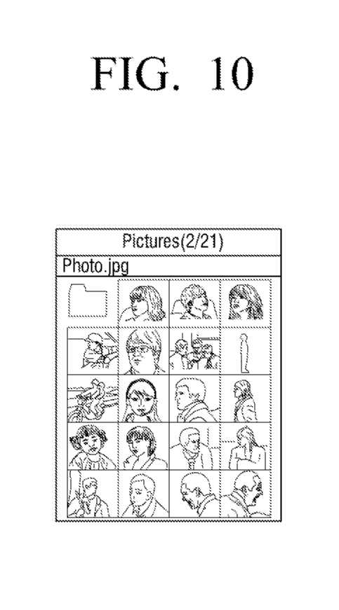

FIG. 10 is a view illustrating a screen when a menu of ‘Pictures’ is displayed according to an exemplary embodiment of the present invention.

Referring to FIG. 10, if the user selects the menu of ‘Pictures’, a list of pictures stored is displayed on the screen.

If a condition for selection of one of the pictures included in the list is prepared, the picture prepared for the selection is highlighted, and, if one of the pictures included in the list is selected, the selected picture is enlarged and displayed. In an exemplary implementation, the selected picture may be enlarged over the entire screen.

The list of pictures may be displayed along with reduced pictures (i.e., thumbnails) or may be displayed along with information regarding titles, sizes, and types of the pictures.

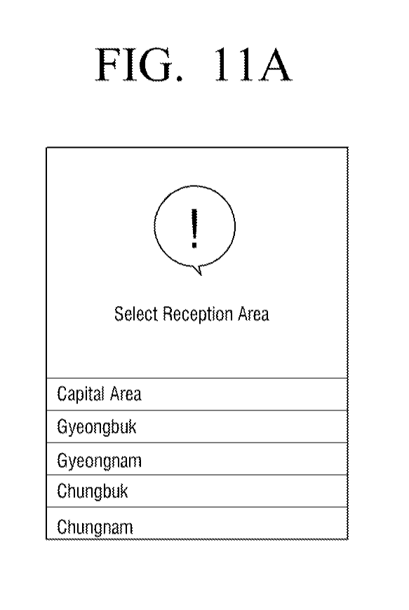

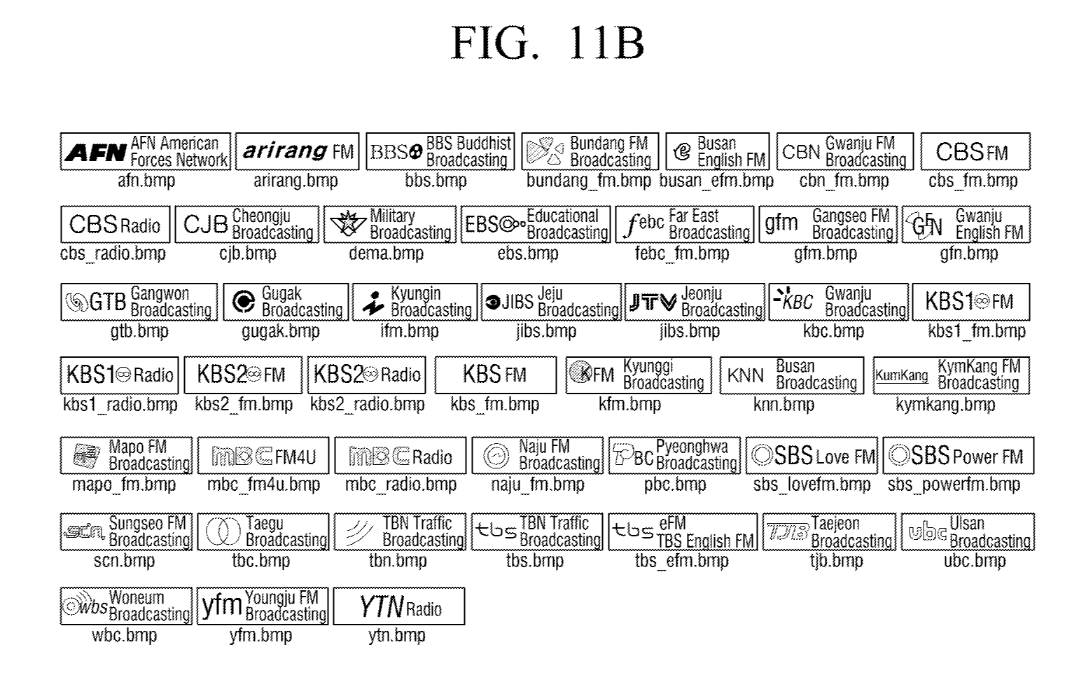

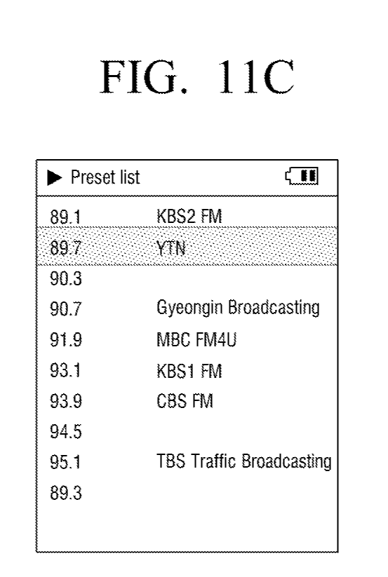

FIGS. 11A to 11C are views illustrating screens when a menu of ‘Settings’ is selected according to an exemplary embodiment of the present invention.

If the user selects the menu of ‘Settings’, the user is able to change setting options regarding the functions corresponding to the sub-menus of the menu of ‘Media’. For example, FIGS. 11A to 11C illustrate a process of setting a frequency at which an FM radio is received.

The MP3 player stores a database containing frequency information of an FM radio broadcasting station for each area and information regarding a logo of a broadcasting station. Accordingly, by selecting a corresponding area, the user can identify frequency information and logo information of broadcasting stations corresponding to an FM radio signal receivable within the corresponding area. FIG. 11A illustrates a screen for selecting an area to receive an FM radio broadcast.

If the area is selected, the frequency information and the logo information of the broadcasting stations corresponding to the FM radio signal receivable within the selected area are extracted as shown in FIG. 11B.

The extracted information includes the frequency information matching with the logo information of the broadcasting station and is displayed as shown in FIG. 11C. If a broadcasting station has no logo, a default logo is displayed for the broadcasting station.

The user selects a desired frequency from the frequency list and receives a broadcast provided from the selected broadcasting station. Also, the user may select a plurality of desired frequencies and create an additional list of broadcasts provided from the selected broadcasting stations.

In the frequency list, an item corresponding to a frequency prepared for selection is highlighted, and if one of the items is selected, a broadcast is received at the frequency corresponding to the selected item, as shown in FIG. 9.

As described above, the process of setting the frequency in order to receive the FM radio is merely an example for convenience of explanation, and setting options for the menus of ‘Music’, ‘Videos’, and ‘Pictures’ may be changed using the menu of ‘Settings’.

### 3. Functions Relating to Menu of ‘Biocheck’

Hereinafter, functions of an MP3 player relating to a menu of ‘Biocheck’ will be explained with reference to FIGS. 12 to 15C.

FIG. 12 is a view illustrating a menu screen when a menu of ‘Biocheck’ is selected according to an exemplary embodiment of the present invention.

Referring to FIG. 12, the menu of ‘Biocheck’ includes three sub-menus of, ‘Fat’, ‘Stress’, and ‘Skin Color’.

As shown in FIG. 12, each sub-menu includes a graphic item and a text item for indicating a type of each sub-menu. For example, the sub-menu of ‘Fat’ includes a graphic item for representing the menu of Fat and a text item (Tat') for representing the menu of ‘Fat’.

As described above, the sub-menu is displayed along with the graphic item and the text item so that the user can more intuitively recognize what type of sub-menu is displayed.

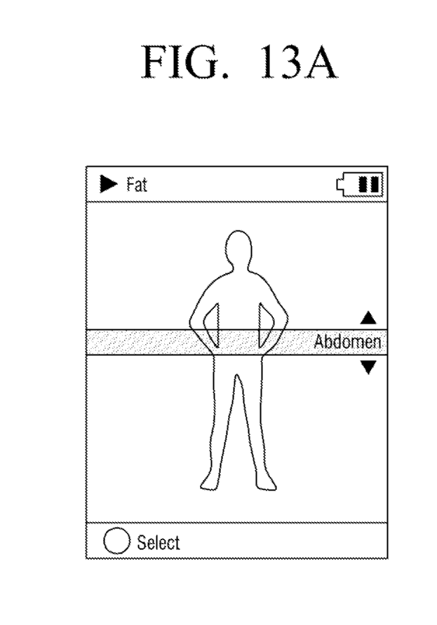

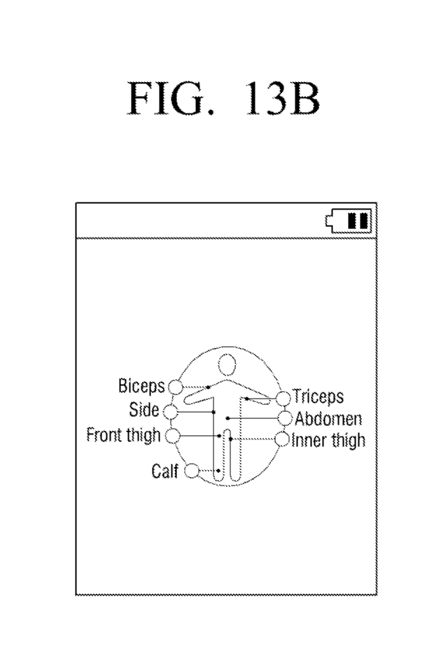

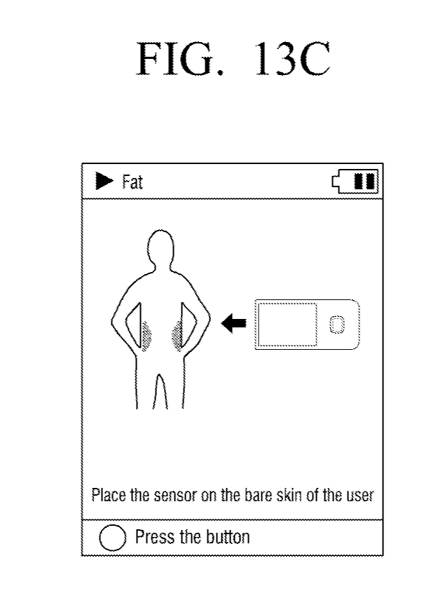

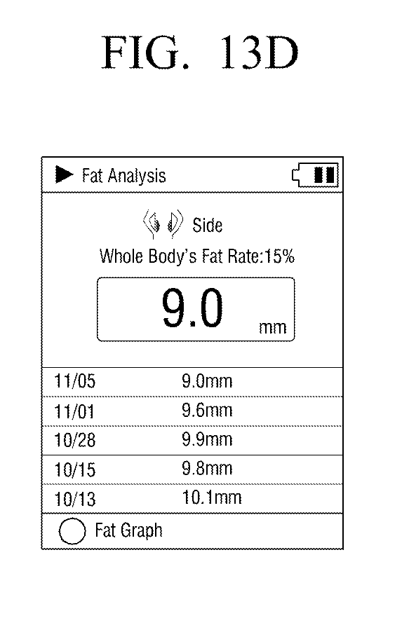

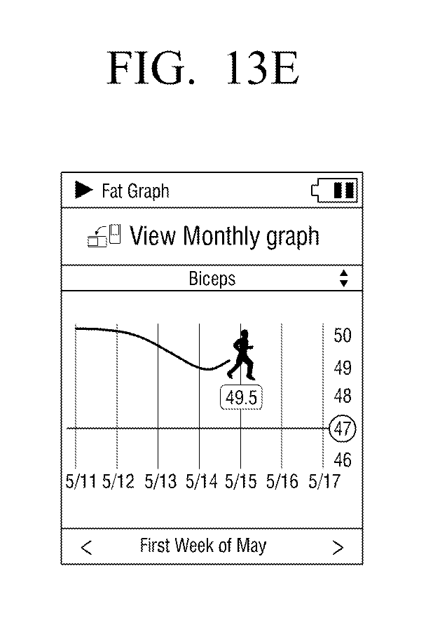

FIGS. 13A to 13E are views illustrating screens when a menu of ‘Fat’ is selected according to an exemplary embodiment of the present invention.

The menu of ‘Fat’ is used for measuring the thickness of subcutaneous fat at each body part of the user or used to measure body fat. If the menu of ‘Fat’ is selected, the user selects a body part for measuring subcutaneous fat or body fat through a vertical manipulation.

For example, if the user wishes to measure subcutaneous fat and body fat of the abdomen, the user selects abdomen on the screen as a part for measuring subcutaneous fat or body fat through the vertical manipulation, as shown in FIG. 13A.

Referring to FIG. 13A, a screen on which a condition prepared for selection of abdomen for measuring subcutaneous fat or body fat is displayed. Under this condition prepared for the section of abdomen, the abdomen of a character displayed on the screen is highlighted and a text (Abdomen) representing that the highlighted portion indicates the user's abdomen is added.

If the user's vertical manipulation is input, the highlight is moved upwardly or downwardly so that another part of the character is highlighted and a text representing what the highlighted part indicates is added.

The reason why the user's body part is set is that subcutaneous fat and body fat are measured in different ways according to the user's body part.

The body parts settable by the user may be provided by the MP3 player. The MP3 player provides information regarding the body parts settable by the user, informs the user which part the user can set, stores information regarding the method of measuring subcutaneous fat and body fat of the body parts in advance, and calculates the subcutaneous fat and the body fat of the selected body part based on the stored information.

Referring to FIG. 13B, the body parts that can be set by the user are displayed. In FIG. 13B, it is possible to set biceps, triceps, side, abdomen, front thigh, inner thigh, and calf. Therefore, the user is able to select one of these body parts and measure the subcutaneous fat or the body fat of the selected body part. Accordingly, the user is able to measure the subcutaneous fat or the body fat according to the body part more accurately.

Referring to FIG. 13C, a screen when the user selects the side is displayed. If the user selects the side in order to measure subcutaneous fat or body fat of the side, the MP3 player displays a guide screen in order for the user to place the upper portion of the MP3 player on the side of the user.

As described above, the upper portion of the MP3 player is provided with diverse optical sensors for measuring a user's subcutaneous fat or body fat. Accordingly, when the user places the upper portion of the MP3 player on his or her side, the MP3 player measures the subcutaneous fat or the body fat of the user's side using the optical sensors.

Referring to FIG. 13D, a resulting value of the measurement is displayed. More particularly, a current resulting value is displayed along with a previous value, so that the user can know how much the subcutaneous fat or the body fat of the user's side changes over time.

Referring to FIG. 13E, the measured resulting value may be displayed in a monthly/daily graph.

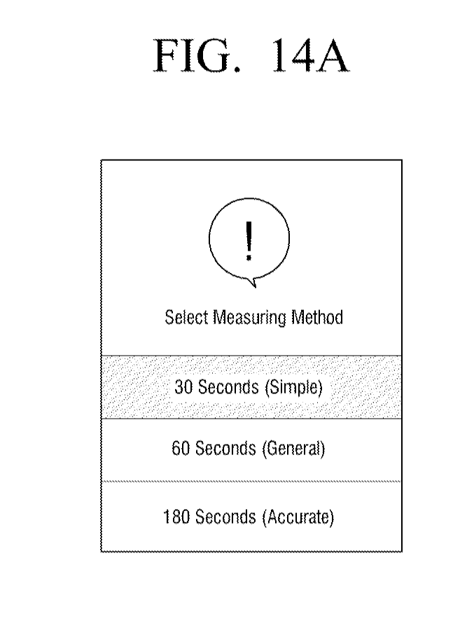

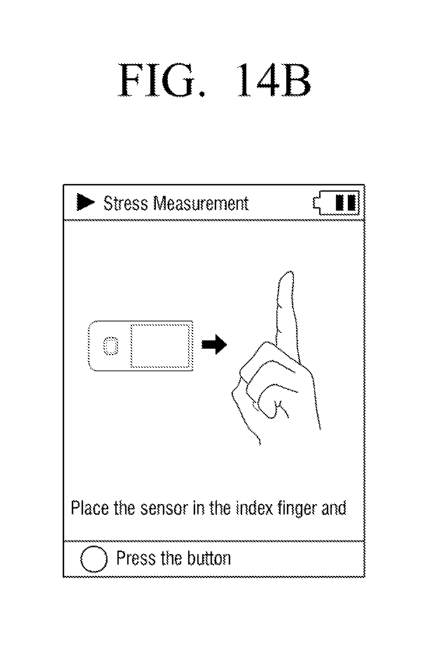

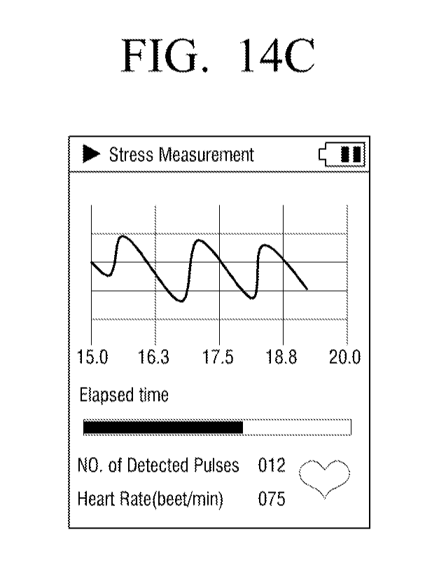

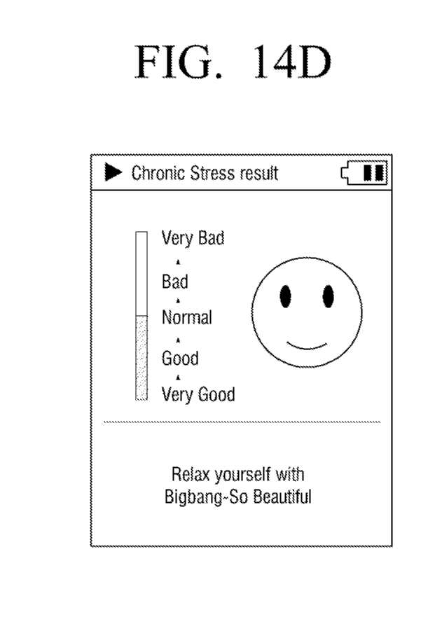

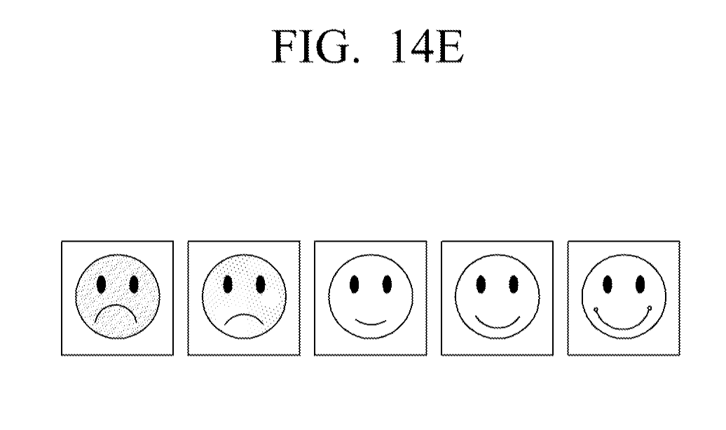

FIGS. 14A to 14E are views illustrating screens when a menu of ‘Stress’ is selected according to an exemplary embodiment of the present invention.

The menu of ‘Stress’ is used for measuring stress of the user of an MP3 player. If the menu of ‘Stress’ is selected, the user is able to select a method for measuring stress through a vertical manipulation.

Referring to FIG. 14A, a screen on which a condition prepared for selection of a ‘simple’ measuring method is displayed. More particularly, under the condition prepared for the selection of a ‘simple’ measuring method, an item corresponding to the ‘simple’ measuring method is highlighted.

Also, the user may select a ‘general’ measuring method or an ‘accurate’ measuring method through a vertical manipulation.

Referring to FIG. 14B, if one of the measuring methods is selected, a guide screen is displayed for the user to place his or her finger on the upper portion of the MP3 player in order to measure stress.

As described above, the various optical sensors are provided on the upper portion of the MP3 player for measuring the user's stress. Accordingly, if the user places his or her finger on the upper portion of the MP3 player, the MP3 player measures the user's heartbeat (heart rate) using the optical sensors and calculates heartbeat or heart rate per minute.

Referring to FIG. 14C, when the stress measurement starts, a progressing graph indicating information of time passing for measurement is displayed along with the detected heartbeat or heart rate.

Referring to FIG. 14D, when the stress measurement finishes, a resulting degree of stress according to the detected heartbeat or heart rate is displayed. The degree of stress may be expressed in a bar-type graph or a smile graphic item, or may be displayed in both of the bar-type graph and the simile graph item.

In other words, if the degree of stress is normal, the bar-type graph and the smile graphic item are displayed, indicating that the degree of stress is normal directly or indirectly.

On the lower portion of the resulting screen, an advising message may be displayed for the user to select whether to listen to music selected based on the measured degree of stress.

The user's health condition or fitness condition is recorded on metadata of a music source while the user is listening to the music. Therefore, the MP3 player extracts a music source that is recorded when the user's health condition or fitness condition is good, and provides the extracted music source to the user, so that the level of stress of the user is changed. For example, if the user's stress index is high, the MP3 player extracts a music source that is recorded when the user's stress index is low and provides the extracted music source to the user, so that the user's stress index is reduced. To achieve this, the MP3 player displays music information regarding the extracted music source on the screen so that the user can select music based on the music information.

In other words, if the user inputs a command to listen to music selected based on the measured stress index, the MP3 player provides the selected music to the user through a speaker or an output port connected to the MP3 player.

Referring to FIG. 14E, the smile graphic items are provided, reflecting five levels of stress index including ‘Very Bad’, ‘Bad’, ‘Normal’, ‘Good’, and ‘Very Good’ from the left to the right.

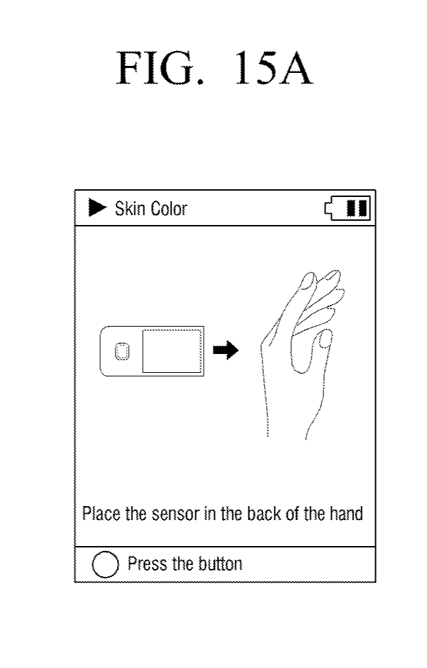

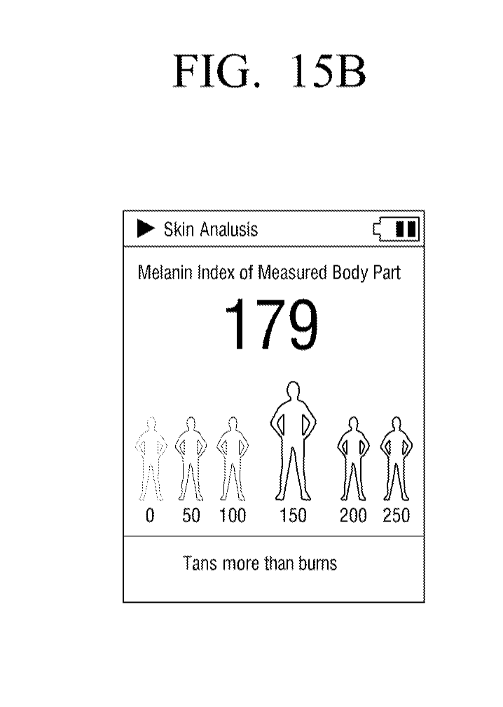

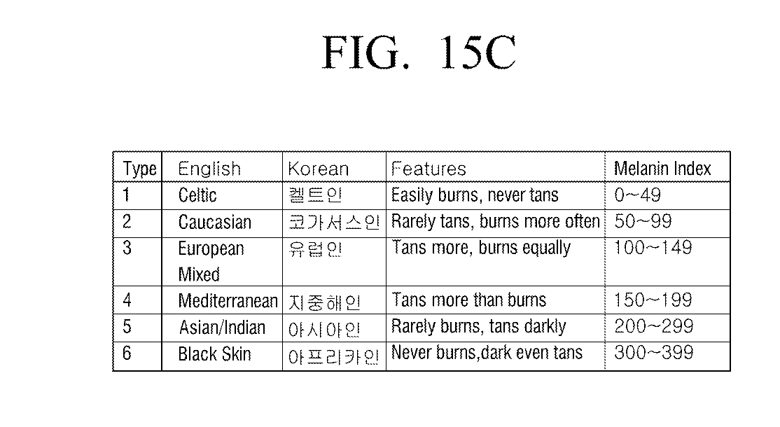

FIGS. 15A to 15C are views illustrating screens when a menu of ‘Skin Color’ is selected according to an exemplary embodiment of the present invention.

Referring to FIG. 15A, the menu of ‘Skin Color’ is used for measuring the user's skin color. If the menu of ‘Skin Color’ is selected, a guide screen is displayed for the user to place the back of his or her hand on the upper portion of the MP3 player in order to measure user's skin color.

As described above, the upper portion of the MP3 player is provided with the various optical sensors for measuring a user's skin color. Accordingly, if the user places the back of his or her hand on the upper portion of the MP3 player, the MP3 player measures a melanin index on the back of user's hand using the optical sensors and displays the measured melanin index on the screen.

Recently, users' interest on beauty is growing and various whitening or tanning products are coming into the market. Accordingly, the menu of ‘Skin Color’ for measuring a user's skin color is provided so that the user can check the effect of these beauty products directly.

If the user wishes to check a melanin index on their face, the user should place the upper portion of the MP3 player on the face.

Referring to FIG. 15B, if the melanin index is measured, a value indicating the measured melanin index is displayed and a character having skin color corresponding to the value is highlighted.

Referring to FIG. 15C, the character may have different skin colors according to the melanin index and may have different features according to the skin color. FIG. 15C illustrates features classified by the melanin index.

Information about the features according to the melanin index is stored in the internal repository of the MP3 player and is provided to the user along with the melanin index, and also may be provided according to a user's extra command.

### 4. Functions Relating to Menu of ‘Fitness’

Hereinafter, the functions of an MP3 player relating to a menu of ‘Fitness’ will be explained with reference to FIGS. 16 to 19.

FIG. 16 is a view illustrating a menu screen when the menu of ‘Fitness’ is selected according to an exemplary embodiment of the present invention.

Referring to FIG. 16, the menu of ‘Fitness’ includes three sub-menus ‘Jogging’, ‘My Coach’, and ‘Pedometer’.

As shown in FIG. 16, each sub-menu includes a graphic item and a text item for indicating a type of each sub-menu. For example, the sub-menu of ‘Jogging’ includes a graphic item and a text item ('Jogging') in order to indicate the menu of ‘Jogging’.

As described above, the graphic item and the text item are also displayed so that the user can more intuitively recognize what type of sub-menu is displayed.

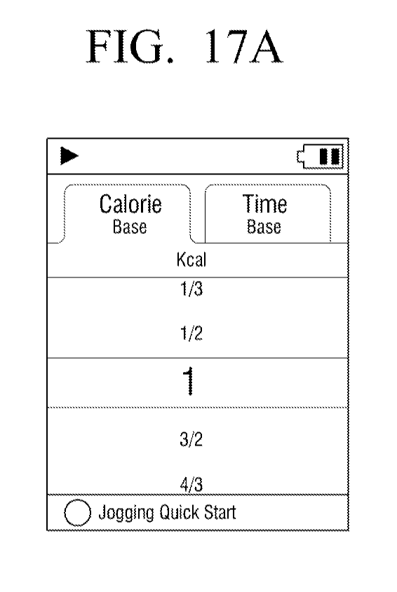

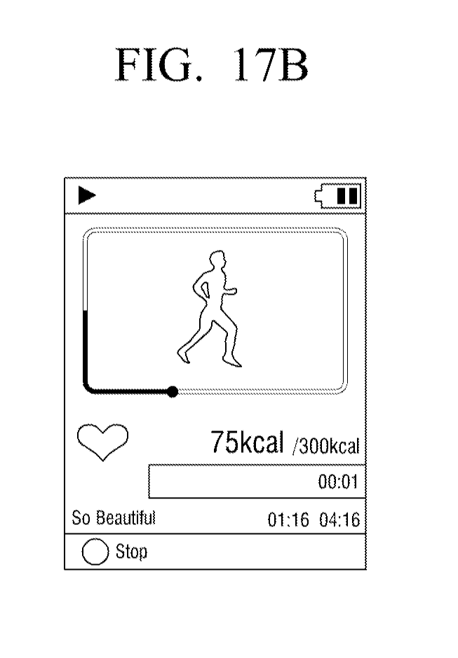

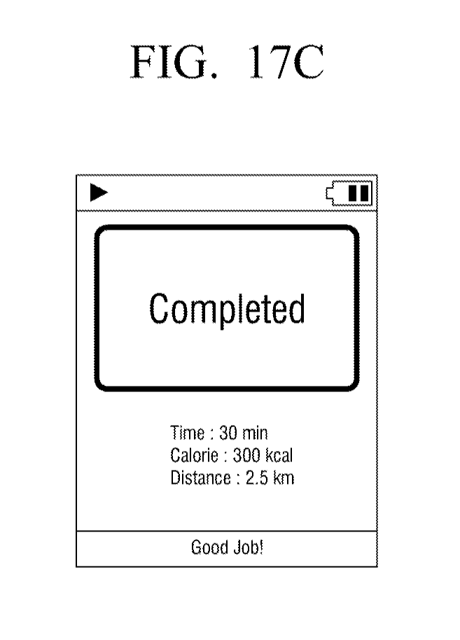

FIGS. 17A to 17C are views illustrating screens when a menu of ‘Jogging’ is selected according to an exemplary embodiment of the present invention.

As described above, the MP3 player may be used by being attached to the user's arm using the armband so that the MP3 player can be used when the user is exercising. When exercising, the user may use the MP3 player to listen to music or check his or her health condition or fitness condition.

The menu of ‘Jogging’ is used for checking and managing the user's fitness condition. If the user selects the menu of ‘Jogging’, a screen for selecting a mode is displayed as shown in FIG. 17A.

The mode is divided into a calorie base mode and a time base mode and one of the two modes is selected by selecting a tab displayed on the upper portion of the screen.

If the user sets a number of target calories to be burned when jogging, the MP3 player, in the calorie base mode, provides information regarding whether the target calories are burned or not and how many calories are burned currently. On the other hand, if the user sets a target time, the MP3 player, in the time base mode, provides information regarding whether the target time is reached or not and how much time elapses currently.

In the calorie based mode, the user may set the target calories to be burned using the screen shown in FIG. 17A. When the user starts jogging after setting the target calories, information regarding calories left until the target calories are burned and currently burned calories is displayed as shown in FIG. 17B. If the user burns the target calories, a message informing that the target calories have been completely burned is displayed on the screen as shown in FIG. 17C.

Also, in the time based mode, the user may set a desired target time. If the user starts jogging after setting the target time, information regarding time left until the target time is reached and the amount of time already passed is displayed on the screen. Also, when the user reaches the target time, a message informing that the target time is reached is displayed on the screen.

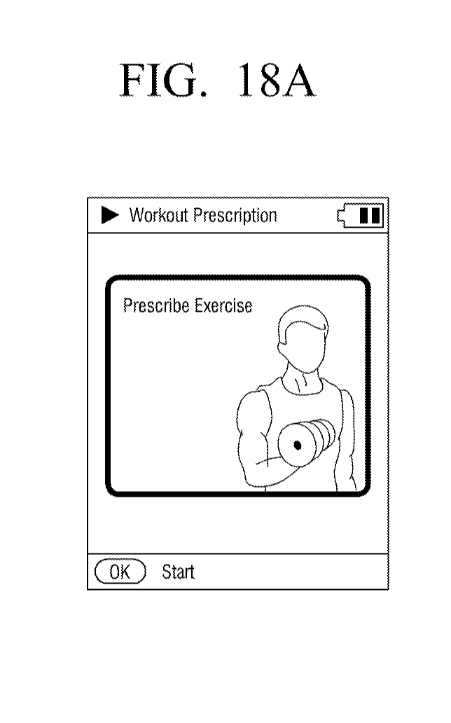

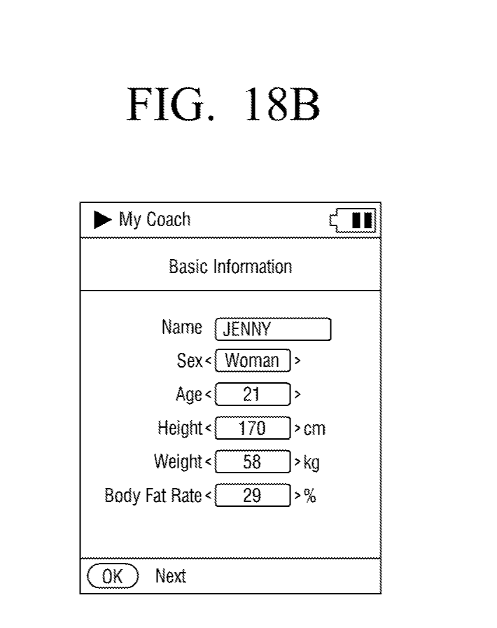

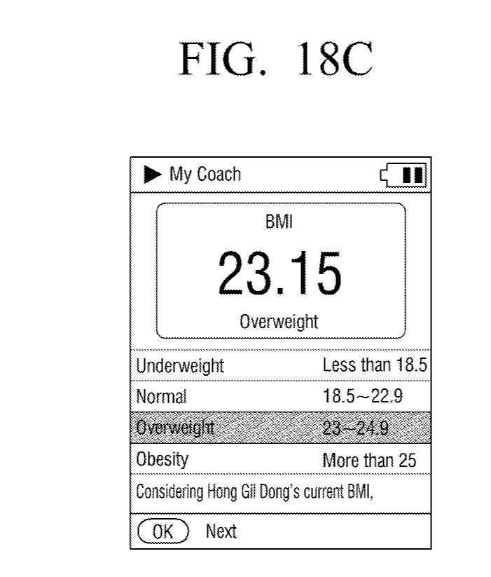

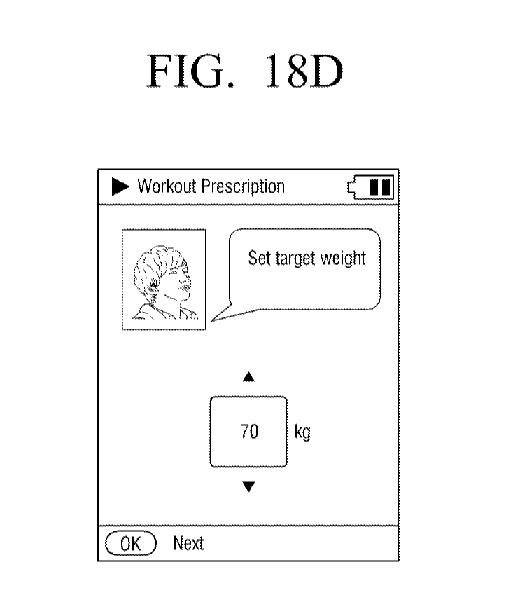

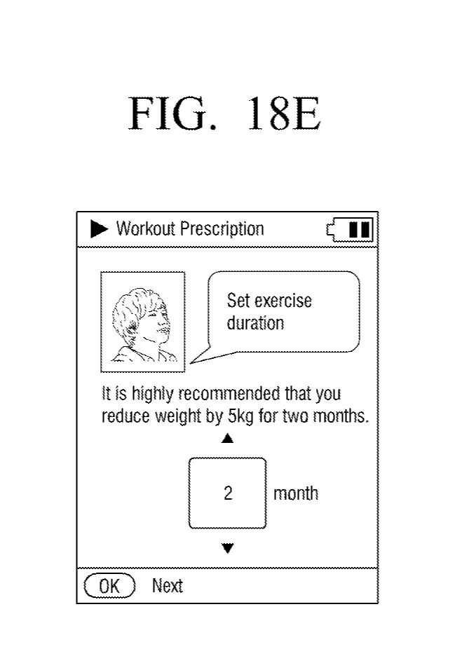

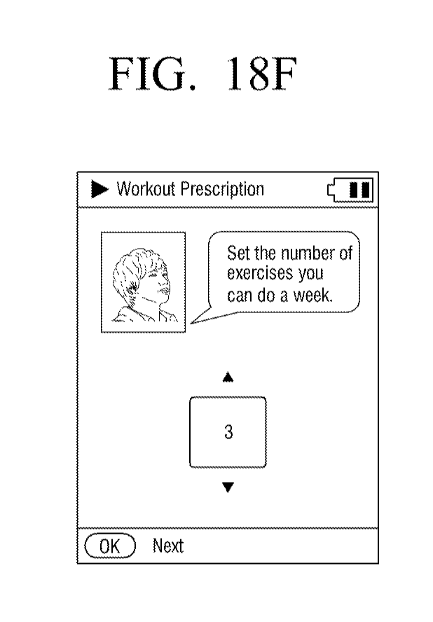

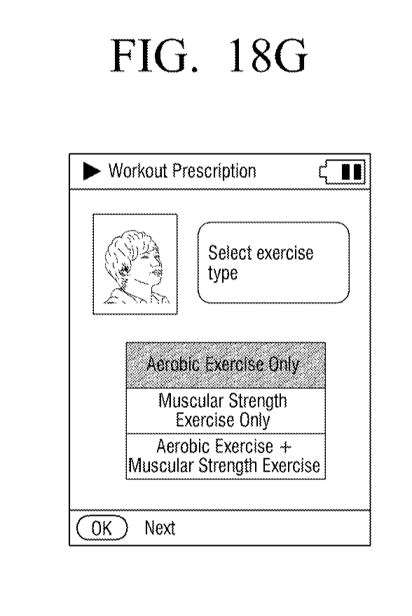

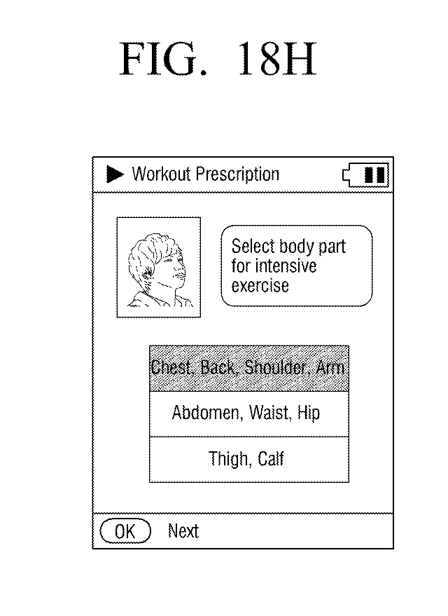

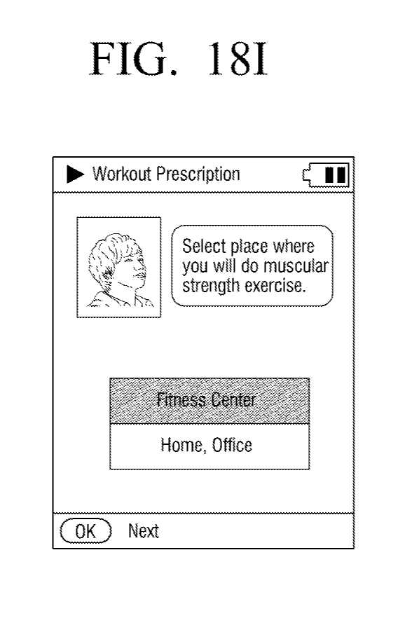

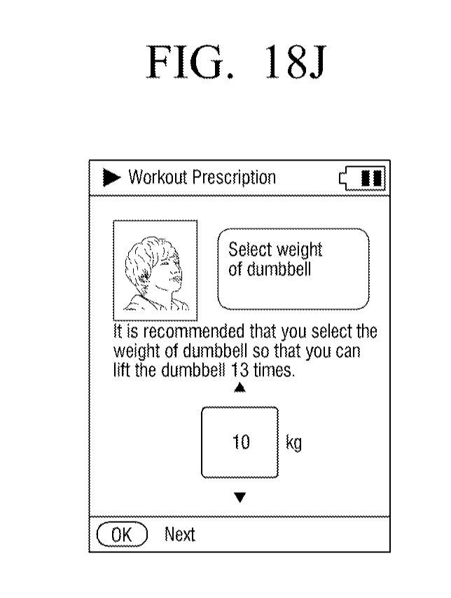

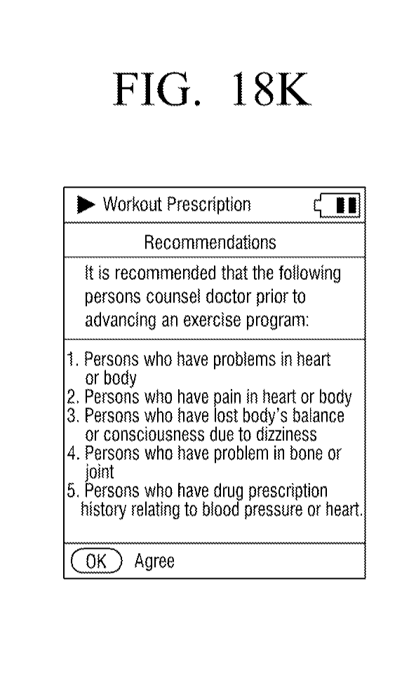

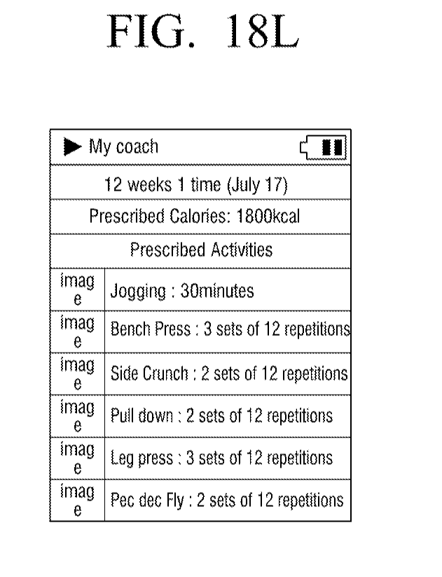

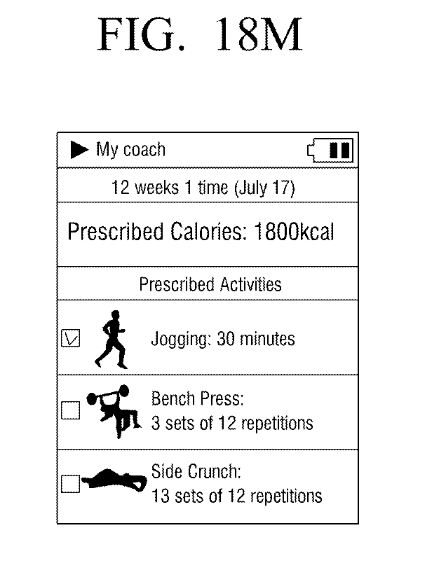

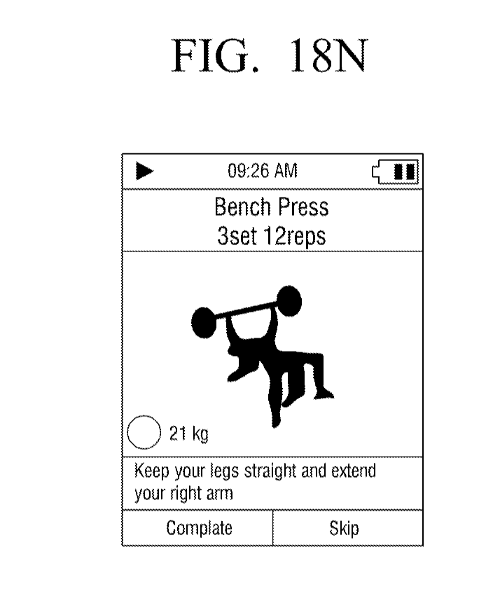

FIGS. 18A to 18N are views illustrating screens when the menu of ‘My Coach’ is selected according to an exemplary embodiment of the present invention.

As described above, the user uses an MP3 player to listen to music or check and manage his/her health condition or fitness condition, while exercising.

The menu of ‘My Coach’ is used for prescribing exercise based on the user's health condition or fitness condition. If the user selects the menu of ‘My Coach’, a screen informing that exercise prescription starts is displayed as shown in FIG. 18A.

Next, a screen for inputting a user's basic information such as name, gender, age, height, weight, and body fat percentage is displayed as shown in FIG. 18B. If the basic information is input from the user, the MP3 player 100 calculates a Body Mass Index (BMI) based on the basic information and provides the BMI to the user as shown in FIG. 18C.

If the user inputs detailed exercise information such as target weight, exercise duration, the number of exercises, preferred activity type, body part, exercise location, and dumbbell weight, in order to receive an effective and accurate exercise prescription, as shown in FIGS. 18D to 18J, a recommendation message concerning what the user needs to be cautious about when exercising is displayed and then exercise is prescribed as shown in FIG. 18K.

Also, on each screen for inputting the detailed exercise information, a guide message, such as ‘It is highly recommended that you reduce weight by 5 kg for two months,” is also displayed so that more appropriate exercise information can be input.

The MP3 player prescribes exercise that the user should do based on the input basic information and the detailed exercise information, as shown in FIG. 18L.

Referring to FIG. 18L, the exercise prescription includes information regarding total calories to be burned, information regarding exercises for burning calories, and information regarding necessary time for exercise or the number of exercises.

The user is able to check the exercise he/she has already done while doing the exercise and, information regarding next exercise is displayed as shown in FIG. 18M. When a user selects a next exercise, information regarding the next exercise may be displayed as shown in FIG. 18N. In the example of FIG. 18N, the number of sets and repetitions of a Bench Press exercise are displayed. Also displayed are the recommended or selected weight for the exercise and advice for properly completing the exercise.

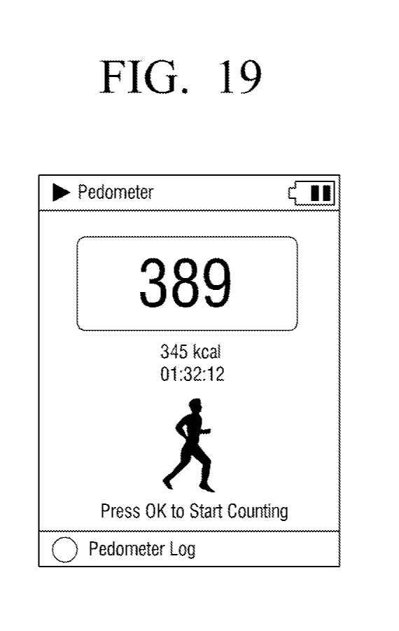

FIG. 19 is a view illustrating a menu screen when a menu of ‘Pedometer’ is selected according to an exemplary embodiment of the present invention.

As described above, the user uses an MP3 player to listen to music or check and manage the user's health condition or fitness condition while exercising.

The menu of ‘Pedometer’ is mainly used for providing the user's fitness condition. More particularly, the menu of ‘Pedometer’ provides a step counter function for counting the number of user's steps. Accordingly, if the user selects the menu of ‘Pedometer’, a screen for counting the number of user's steps is displayed as shown in FIG. 19.

The number of user's steps is counted using the aforementioned 3-axis sensor. For example, if the MP3 player detects repeated movement in a vertical direction by the 3-axis sensor, the MP3 player determines this movement as one step, thereby counting the number of user's steps.

### 5. Functions Relating to the Menu of ‘My Log’

Hereinafter, the functions of an MP3 player relating to a menu of ‘My Log’ will be explained with reference to FIGS. 20 to 24C.

FIG. 20 is a view illustrating a menu screen when the menu of ‘My Log’ is displayed according to an exemplary embodiment of the present invention.

Referring to FIG. 20, if the menu of ‘My Log’ is selected, a user's budgeted calories for one day are displayed. Accordingly, the user can know the amount of calories the user can eat for one day or target calories of food that the user should eat for one day.

Also, if the menu of ‘My Log’ is displayed, information regarding sub-menus is displayed along with the budgeted calories.

As shown in FIG. 20, the menu of ‘My Log’ includes five sub-menus, ‘Today's Summary,’ Exercise Diary', ‘Food Diary’, ‘Calorie Manager’, and ‘My Info’. Each sub-menu includes a text item indicating a type of each sub-menu.

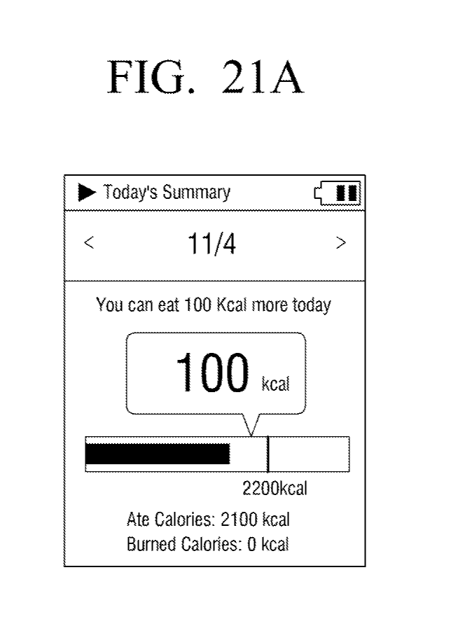

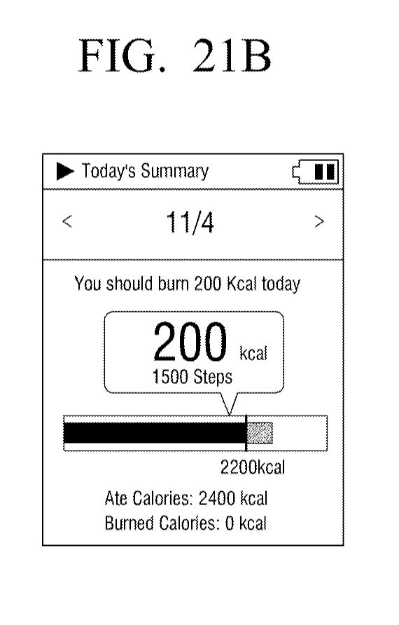

FIGS. 21A and 21B are views illustrating screens when a menu of ‘Today's Summary’ is selected according to an exemplary embodiment of the present invention. More particularly, information regarding the budgeted calories and burned calories is displayed on the screens of FIGS. 21A and 21B.

If the burned calories are less than the budgeted calories, the screen of FIG. 21A is displayed. Referring to FIG. 21A, today's budgeted calories are 2200Kcal but the calories actually burned are 2100Kcal. Therefore, the user is made aware that the user can eat more food of 100Kcal.

Accordingly, the user can intuitively obtain information regarding the amount of food that the user ate and the amount of food that the user will eat.

If the budgeted calories are greater than the burned calories, the screen of FIG. 21B is displayed. Referring to FIG. 21B, today's budgeted calories are 2200Kcal but 2400Kcal were eaten.

Also, a way to burn the excess calories may be also displayed. In other words, the MP3 player may display the information regarding the amount of excess calories and a manner in which to burn the excess calories, so that the user can know that he or she ate food in excess of the budgeted calories and also is urged to do exercise to burn the excess calories.

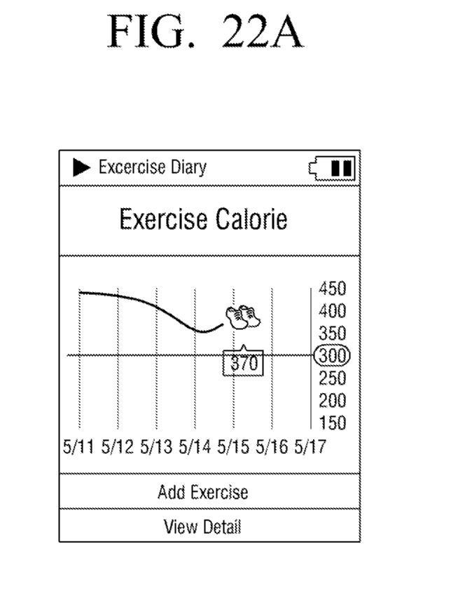

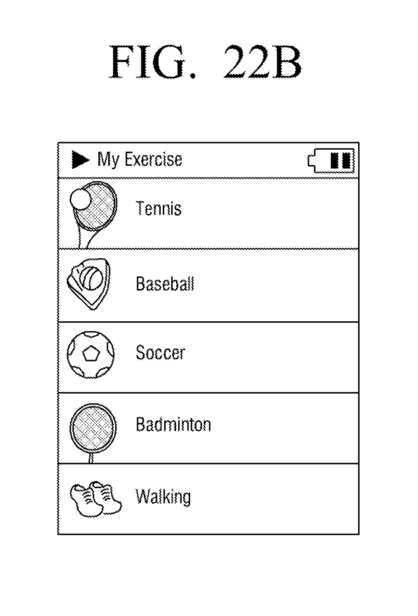

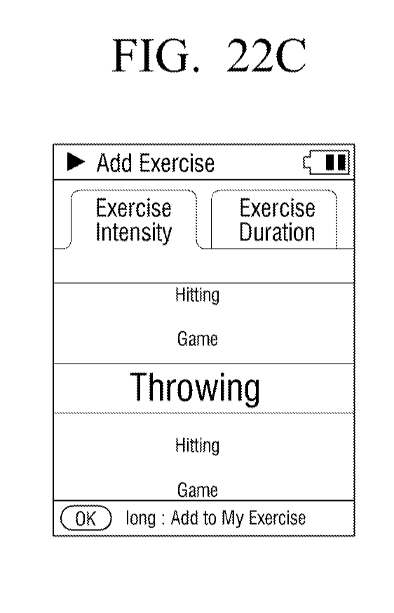

FIGS. 22A to 22C are views illustrating screens when a menu of ‘Exercise Diary’ is selected according to an exemplary embodiment of the present invention.

Referring to FIG. 22A, the menu of ‘Exercise Diary’ is used for calculating and managing calories that a user burns. If the user selects the menu of ‘Exercise Diary’, the number of burned calories is displayed according to date in a graph.

If the user burns calories by doing exercise or if the user wishes to burn more calories by doing exercise, the user may add an activity that the user has done or will do, as shown in FIG. 22B or 22C.

In this case, calorie information regarding each activity is stored in the internal repository of the MP3 player so that the user can burn more calories corresponding to the activity selected by the user.

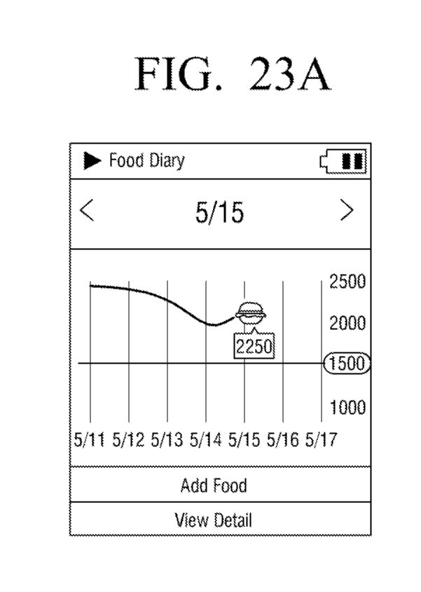

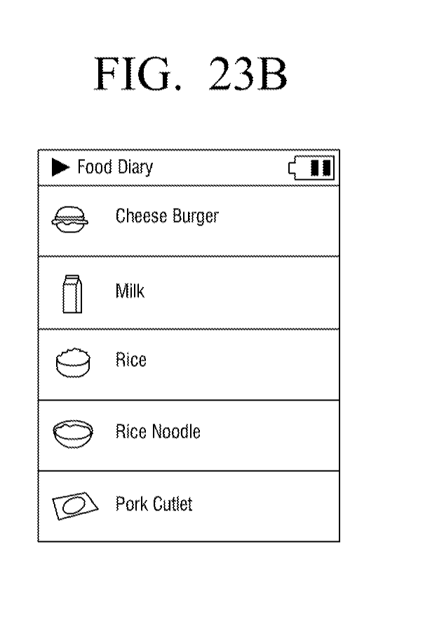

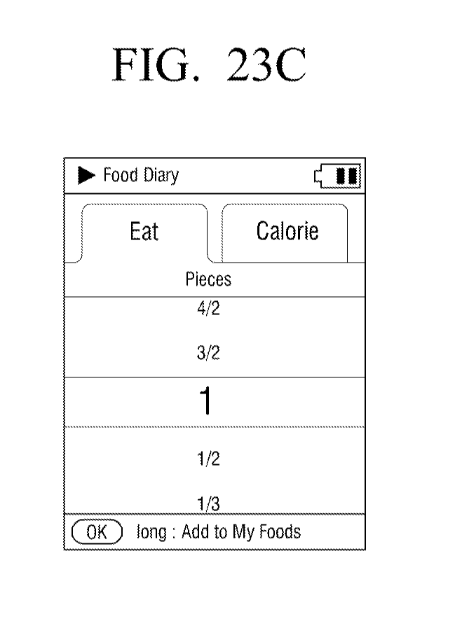

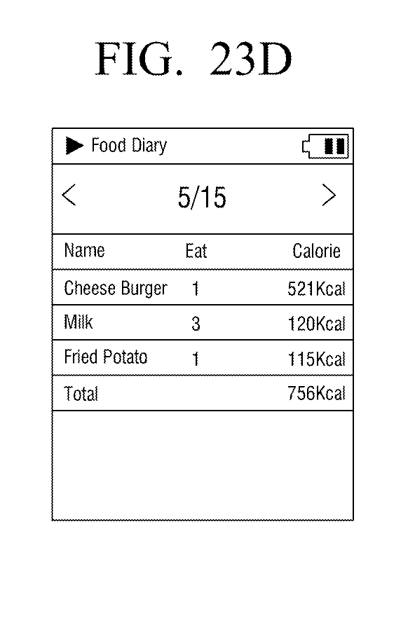

FIGS. 23A to 23D are views illustrating screens when a menu of ‘Food Diary’ is selected according to an exemplary embodiment of the present invention.

Referring to FIG. 23A, the menu of ‘Food Diary’ is used for calculating and managing calories of food that the user ate. If the user selects the menu of ‘Food Diary’, calories that the user ate are displayed according to date in a graph.

If the user wishes to eat more food or if the user ate more food, the user may add food to eat or food that the user already ate using the screens of FIGS. 23B and 23C. More specifically, the user selects food using a food selection screen shown in FIG. 23B and selects an amount of food that the user ate using an amount selection screen shown in FIG. 23C.

In this case, calorie information of each food is stored in the internal repository of the MP3 player so that the calories corresponding to the food selected by the user are added to the calories that the user ate.

Referring to FIG. 23D, if the food to be eaten or the food that the user ate is added, total calories that the user ate for one day is calculated and provided to the user.

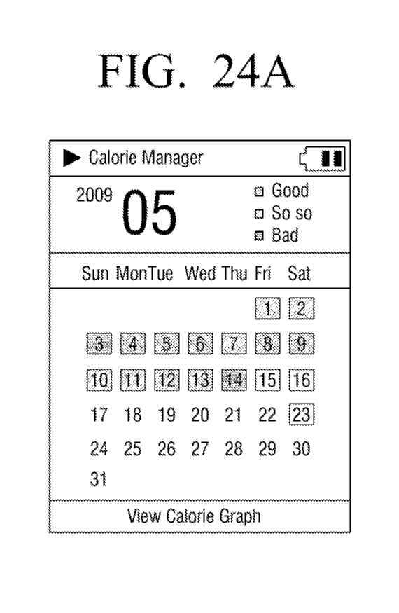

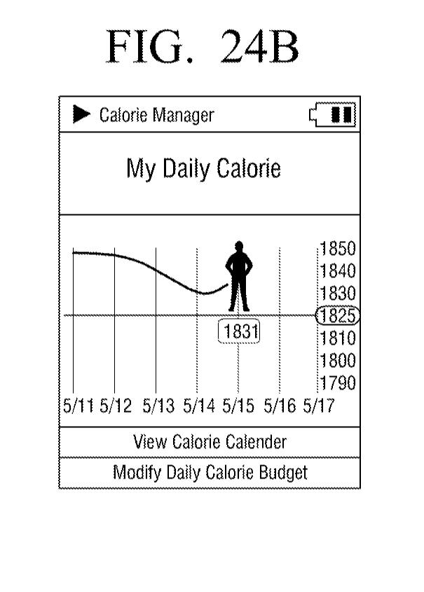

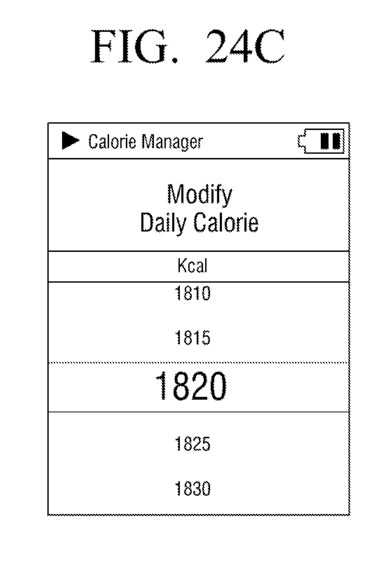

FIGS. 24A to 24C are views illustrating screens when a menu of ‘Calorie Manager’ is selected according to an exemplary embodiment of the present invention.

The menu of ‘Calorie Manager’ is used for calculating and managing a daily calorie burning state, based on the information regarding a user's target calories to be burned, calories that the user ate, and calories that the user actually burns. If the user selects the menu of ‘Calorie Manager’, the information regarding the calorie burning state according to date may be discriminated by color and displayed in a calendar format as shown in FIG. 24A, or displayed in a graph as shown in FIG. 24B.

Also, the MP3 player may modify the target calories based on the calorie burning state according to date, and may display the modified target calories as shown in FIG. 24C.

### 6. Generating and Using Tagged Information

Hereinafter, a method for generating and using tagged information will be explained with reference to FIGS. 25 to 27E.

As described above, a user is able to replay content by manipulating the menu of ‘Media’ and the sub-menus thereof, and check and manage the user's health condition or fitness condition by manipulating the menus of ‘Biocheck’ and ‘Fitness’ and the sub-menus thereof.

The functions may be performed independently or two or more functions may be performed simultaneously. For example, the user may replay a moving picture file by manipulating the menu of ‘Media’, and measure his or her stress index by manipulating the menu of ‘Biocheck’ while viewing the moving picture. Also, the user may replay a music file by manipulating the menu of ‘Media’ and receive an exercise prescription by manipulating the menu of ‘Fitness’ while listening to the replayed music.

Also, even in the absence of an extra menu manipulation, an MP3 player is able to check the user's health condition or fitness condition automatically. For example, when the user is jogging while listening to music by replaying a music file, the MP3 player checks the user's fitness condition at that time. Also, the checked health condition or fitness condition is tagged onto the music and stored.

This will be explained in more detail with reference to FIG. 25.

FIG. 25 is a view illustrating a music file which is tagged with condition information about user's health condition or fitness condition according to an exemplary embodiment of the present invention.

Referring to FIG. 25, the user burns 75Kcal by jogging 2 km for 7 minutes and 30 seconds when listening to the music titled ‘So Beautiful’. A stress index at that time is normal.

As described above, since the user's condition information when the user listens to specific music is tagged onto a corresponding music file and stored, the user can know his or her health condition or fitness condition when the user listens to the music.

The MP3 player selects music that the user needs based on the condition information and creates a play list containing the selected music and provides the play list to the user. More particularly, the MP3 player selects music that is tagged with condition information of a higher level than that of current condition information, and lists the selected music and provides the list to the user.

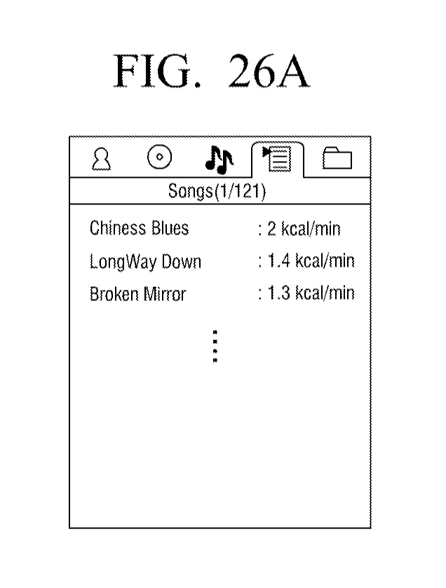

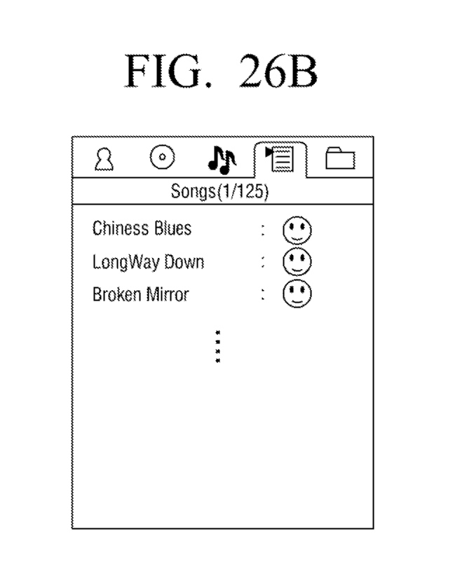

This will be explained with reference to FIGS. 26A and 26B.

FIGS. 26A and 26B are views illustrating a method for using tagged information according to an exemplary embodiment of the present invention.

As described above, if the user manipulates the menu of ‘Jogging’ of the menu of ‘Fitness’, the MP3 player checks and manages the user's fitness condition.

Specifically, the MP3 player analyzes an existing jogging record of the user, extracts the best music files for jogging, creates a play list based on the extracted music files, and provides the play list to the user.

In other words, as shown in FIG. 26A, the MP3 player selects music that achieves the highest calorie burn per second, and provides a play list containing the selected music to the user so that the user can listen to the best music for jogging.

Also, as described above, if the menu of ‘Stress’ of the menu of ‘Biocheck’ is manipulated, the user can check the user's stress index using the MP3 player.

The MP3 player extracts the best music files for stress relief based on the analyzed stress index, creates a play list based on the extracted music files, and provides the play list to the user.

In other words, as shown in FIG. 26B, the music that reduces the stress index when the user listens to it is selected and a play list of the music is provided to the user, so that the user can enjoy the best music for stress relief.

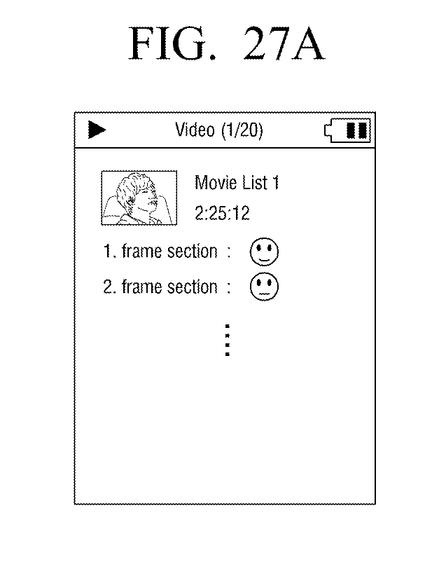

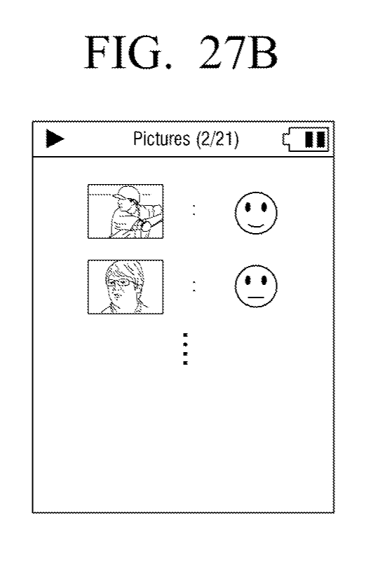

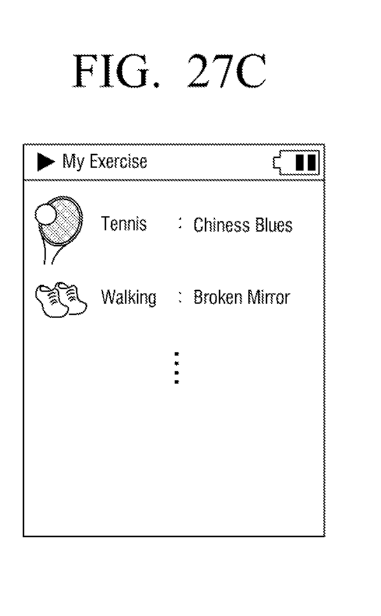

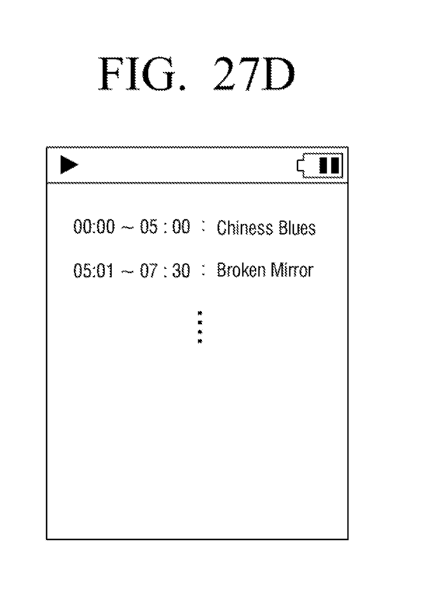

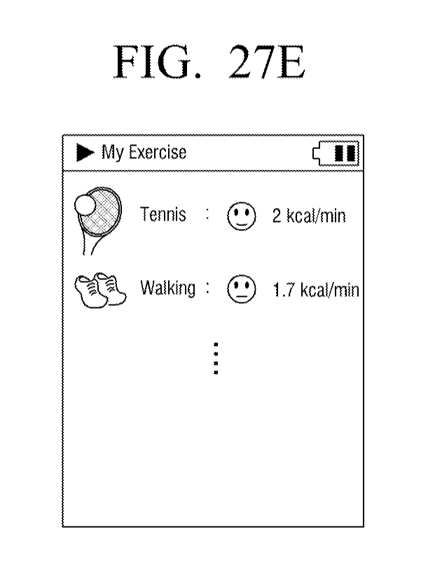

FIGS. 27A to 27E are views illustrating a method of using tagged condition information according to an exemplary embodiment of the present invention.

In this exemplary embodiment, it should be noted that user's condition information is tagged onto not only a music file but also to a moving picture file or a picture file. More particularly, in the case of a moving picture file, the user's condition information may be extracted from every frame section and tagged. In the case of a picture file, the user's condition information may be extracted from every picture file and tagged.

This operation is illustrated in FIGS. 27A and 27B. FIG. 27A illustrates that the user's stress index is extracted from every frame section of a specific moving picture file and tagged, and FIG. 27B illustrates that the user's stress index is extracted from every single picture and tagged.

The tagged information, such as condition information about the health condition or fitness condition is used for creating the play list for the content, as described above. However, based on the replayed content, an exercise type or a suitable jogging time may be recommended. In this case, as described above, the exercise type or the jogging time may be tagged onto the content or a title of the content may be tagged onto the exercise type or the jogging time.

This operation is illustrated in FIGS. 27C and 27D. More particularly, FIG. 27C illustrates that music suitable for a corresponding activity is tagged onto that activity, and FIG. 27D illustrates that suitable music is tagged onto exercise time.

The tagged information may not necessarily be the user's information when the content is replayed. For example, the stress index or the calories burned per second according to an activity selected by the user may be tagged to the activity, and a suitable activity may be recommended based on the information tagged according to the stress index or necessary calories.

This operation is illustrated in FIG. 27E. More specifically, FIG. 27E illustrates that the stress index and the calories burned per second measured according to the activities are tagged onto the activities.

The foregoing explanations are merely exemplary for convenience of explanation. Any other method of using tagged information may fall within the range of the present disclosure. This will be applied to the following explanation.

### 7. Flowchart for Providing Play List

FIG. 28 is a flowchart illustrating a method for providing a play list according to an exemplary embodiment of the present invention.

It is determined in step S200 if a replay command regarding content is input. If it is determined in step S200 that a replay command regarding content is input, the MP3 player replays the content according to the input replay command in step S210, and measures condition information of a user when the corresponding content is replayed in step S220.

The MP3 player tags the corresponding content with the measured condition information in step S230.

In step S240, it is determined if the user's condition is re-measured. If it is determined in step S240 that the user's condition information is re-measured, the MP3 player extracts content for leveling up the re-measured condition information in step S270.

The contents are extracted with reference to the condition information tagged onto the contents. For example, if the user's stress index is considered as being high based on the re-measured condition information, the MP3 player extracts the contents that are tagged with condition information indicating a low stress index.

If such contents are extracted, the MP3 player creates a list containing the extracted contents and displays the list in step S290, and replays a content selected from the list by the user in step S300.

If it is determined in step S240 that the user's condition information is not re-measured, it is determined if an exercise start command is input from the user through a key provided on the MP3 player in step S250 or if an exercise start is sensed by the 3-axis acceleration sensor provided on the MP3 player in step S260. If it is determined in step S250 that an exercise start command is input from the user through a key provided on the MP3 player or if it is determined in step S260 that an exercise start is sensed by the 3-axis acceleration sensor provided on the MP3 player, the MP3 player extracts contents for leveling up the condition information when the user does exercise in step S280.

The contents are extracted with reference to condition information tagged onto the contents. For example, if the user's condition information is considered as being high when the user views or listens to a specific content while doing exercise, the MP3 player extracts contents that are tagged with the condition information determined as high when the user does exercise.

If such contents are extracted, the MP3 player generates a play list containing the extracted contents and displays the play list in step S290, and replays a content selected from the list by the user in step S300.

### 8. Configuration of MP3 Player

Hereinafter, a configuration of an MP3 player will be explained with reference to FIGS. 29 and 30.

FIG. 29 is a block diagram illustrating an MP3 player according to an exemplary embodiment of the present invention.

Referring to FIG. 29, the MP3 player 100 includes a sensor module 110, a multimedia function block 120, a Graphic User Interface (GUI) generator 130, a display 140, a controller 150, a user command receiver 160, and a storage unit 170, in order to replay multimedia and check and manage user's health condition, fitness condition and schedule.

The sensor module 110 measures the user's health condition and fitness condition. More particularly, the sensor module 110 includes optical sensors 111, 113, 115, 117 for measuring body fat, stress, skin color, and heart rate, and a 3-axis acceleration sensor 119 for measuring burned calories.

Hereinafter, the sensor module 110 will be explained in more detail with reference to FIG. 30.

FIG. 30 is a view illustrating a sensor module according to an exemplary embodiment of the present invention.

Referring to FIG. 30, the sensor module 110 includes the first optical sensor 111, the second optical sensor 113, the third optical sensor 115, and the fourth optical sensor 117, and the 3-axis acceleration sensor 119. More particularly, the first optical sensor 111, the second optical sensor 113, the third optical sensor 115, and the fourth optical sensor 117 are provided on a lower end portion of the sensor module 110 in parallel, and the 3-axis acceleration sensor 119 is built into the sensor module 110.

The first optical sensor 111 includes photo diodes and is used for measuring subcutaneous fat and body fat, skin color, stress, and heart rate.

The second optical sensor 113 includes two Light Emitting Diodes (LEDs) emitting light of a wavelength of 660 nm and two LEDs emitting light of a wavelength of 880 nm, and is used for measuring subcutaneous fat and body fat, and skin color.

The third optical sensor 115 includes an LED emitting light of a wavelength of 940 nm and is used for measuring stress and heart rate.

The fourth optical sensor 117 includes an LED emitting light of a wavelength of 880 nm, and is used for measuring subcutaneous fat and body fat.

In other words, the first optical sensor 111, the second optical sensor 113, and the fourth optical sensor 117 are used for measuring subcutaneous fat and body fat, the first optical sensor 111 and the second optical sensor 113 are used for measuring skin color, and the first optical sensor 111 and the third optical sensor 115 are used for measuring stress or heart rate.

More particularly, the first optical sensor 111, the second optical sensor 113, and the fourth optical sensor 117 irradiate light onto a user's skin and determine the user's subcutaneous fat and body fat, and skin color according to an intensity of light reflected from the user's skin.

Also, the first optical sensor 111 and the third optical sensor 115 irradiate light onto the user's skin and determine the user's stress and heart rate according change in the intensity of light reflected from the user's skin.

Also, the 3-axis acceleration sensor 119 detects acceleration and vibration by detecting motion in X-axis, Y-axis, and Z-axis directions, and is used for measuring the number of user's steps. Based on the number of steps, the user's burned calories are calculated.

Referring again to FIG. 1, the sensor module 110 transmits sensed information to the controller 150.

The multimedia function block 120 displays screens according to user's manipulations. More particularly, the multimedia function block 120 replays contents such as a moving picture, a still picture, music, and text, or executes contents, programs, and applications relating to user's health condition, fitness condition, and schedule, in order to display screens according to user's manipulations.

The GUI generator 130 generates a GUI such as items and adds the GUI to the contents replayed by the multimedia function block 130 or the applications or programs executed or operating by the multimedia function block 110.

The display 140 displays the contents replayed by the multimedia function block 110 and the applications or programs executed by the multimedia function block 110, and the GUI generated by the GUI generator 130.

The controller 150 controls the sensor module 110 and the multimedia function block 120 to perform their respective functions according to the user's manipulation input through the user command receiver 160.

More particularly, the controller 150 controls the multimedia function block 120 to replay the content corresponding to the manipulation of the menu of ‘Media’.

Also, the controller 150 controls the sensor module 110 and the multimedia function block 120 to sense the user's health condition and execute the corresponding application or program according to the manipulation of the menu of ‘Biocheck’.

Also, the controller 150 controls the sensor module 110 and the multimedia function block 120 to sense the user's health condition and execute the corresponding application or program according to the manipulation of the menu of ‘Fitness’.

Also, the controller 150 controls the multimedia function block 120 to execute the corresponding application or program according to the manipulation of the menu of ‘My Log’.

Also, the controller 150 controls the multimedia function block 120 to execute the corresponding application or program according to the manipulation of the menu of ‘Health Application”.

The user command receiver 160 receives a user's command input through the aforementioned keys provided on the front and side panels of the MP3 player 100 and transmits the user's command to the controller 150.

The storage unit 170 may be realized as a Hard Disk Drive (HDD) or a memory, and stores programs for operating the MP3 player 100 and various contents and applications. Also, the storage unit 170 stores information for converting values measured by the sensor module 110 into body fat rate, stress index, melanin index, or heart rate.

While the invention has been shown and described with reference to certain exemplary embodiments thereof, it will be understood by those skilled in the art that various changes in form and details may be made therein without departing from the spirit and scope of the invention as defined by the appended claims and their equivalents.

What is claimed is:

1. A method for tagging condition information, the method comprising:
if a content selected from a plurality of contents is being played, measuring condition information of a user; and
tagging the content with the measured condition information.

2. The method as claimed in claim 1, further comprising:
re-measuring condition information of the user; and
extracting at least one content tagged with specific condition information based on the re-measured condition information and replaying the extracted content.

3. The method as claimed in claim 2, further comprising:
generating a content list containing the extracted at least one content and providing the content list.

4. The method as claimed in claim 3, wherein the content list contains information regarding the at least one content and condition information tagged onto each content.

5. The method as claimed in claim 2, wherein the specific condition information includes condition information having a level higher than a level of the re-measured condition information.

6. The method as claimed in claim 1, further comprising:
if at least one of a specific command is input from the user and a specific motion of the user is detected, extracting at least one content from the plurality of contents based on the condition information tagged onto the content and replaying the extracted content.

7. The method as claimed in claim 6, wherein the specific command includes an exercise start command of the user,
and further wherein the specific motion includes an exercise start motion of the user that is sensed by a 3-axis acceleration sensor.

8. The method as claimed in claim 1, wherein the condition information includes information regarding at least one of subcutaneous fat, body fat, stress, skin color, and a heart rate of the user.

9. The method as claimed in claim 8, wherein the subcutaneous fat, body fat, stress, skin color, and the heart rate of the user are calculated based on values measured by an optical sensor.

10. The method as claimed in claim 1, wherein the condition information includes information regarding a number of steps of the user counted from a time at which replay of the selected content starts to a time at which the replay finishes.

11. A multimedia apparatus comprising:
a sensor module for, if a content selected from a plurality of contents is being played, measuring condition information of a user; and
a controller for tagging the content with the measured condition information.

12. The multimedia apparatus as claimed in claim 11, wherein, if condition information of the user is re-measured by the sensor module, the controller extracts at least one content tagged with specific condition information based on the re-measured condition information, and replays the extracted content.

13. The multimedia apparatus as claimed in claim 12, wherein the controller generates a content list containing the extracted at least one content and provides the content list.

14. The multimedia apparatus as claimed in claim 13, wherein the content list contains information regarding the at least one content and condition information tagged onto each content.

15. The multimedia apparatus as claimed in claim 12, wherein the specific condition information includes condition information having a level higher than a level of the re-measured condition information.

16. The multimedia apparatus as claimed in claim 11, further comprising:
a user command receiver for receiving a manipulation command,
wherein, if at least one of a specific command is input from the user and a specific motion of the user is detected by the sensor module, the controller extracts at least one content from the plurality of contents based on the condition information tagged onto the content and replays the extracted content.

17. The multimedia apparatus as claimed in claim 16, wherein the specific command includes an exercise start command of the user,
and further wherein the specific motion includes an exercise start motion of the user that is sensed by a 3-axis acceleration sensor.

18. The multimedia apparatus as claimed in claim 11, wherein the condition information includes information regarding at least one of subcutaneous fat, body fat, stress, skin color, and a heart rate of the user.

19. The multimedia apparatus as claimed in claim 18, wherein the subcutaneous fat, body fat, stress, skin color, and the heart rate of the user are calculated based on values measured by an optical sensor included in the sensor module.

20. The multimedia apparatus as claimed in claim 11, wherein the condition information includes information regarding a number of steps of the user counted from a time at which replay of the selected content starts to a time at which the replay finishes.
